(19) United States
(12) Patent Application Publication
(10) Pub. No.: US 2007/0178501 A1
(43) Pub. Date: Aug. 2, 2007

(54) System and method for integrating and validating genotypic, phenotypic and medical information into a database according to a standardized ontology

(76) Inventors: Matthew Rabinowitz, Portola Valley, CA (US); Jonathan Ari Sheena, San Francisco, CA (US); Zachary Paul Demko, Somerville, MA (US); Christopher Clark, Beijing (CN); Nigam Shah, Belmont, CA (US)

Correspondence Address: ZACHARY P DEMKO, 31B SAINT JAMES AVE, SOMERVILLE, MA, 02144, US

(21) Appl. No.: 11/634,550

(22) Filed: Dec. 6, 2006

Related U.S. Application Data
(60) Provisional application No. 60/742,305, filed on Dec. 6, 2005.
(60) Provisional application No. 60/754,396, filed on Dec. 29, 2005.
(60) Provisional application No. 60/774,976, filed on Feb. 21, 2006.
(60) Provisional application No. 60/789,506, filed on Apr. 4, 2006.
(60) Provisional application No. 60/817,741, filed on Jun. 30, 2006.
(60) Provisional application No. 60/846,589, filed on Sep. 22, 2006.
(60) Provisional application No. 60/846,610, filed on Sep. 22, 2006.

(51) Int. Cl. C12Q 1/68; G06F 19/00; G06Q 50/00

(52) U.S. Cl. 435/6; 702/20; 705/3

(57) ABSTRACT

The system described herein enables clinicians and researchers to use aggregated genetic and phenotypic data from clinical trials and medical records to make the safest, most effective treatment decisions for each patient. This involves (i) the creation of a standardized ontology for genetic, phenotypic, clinical, pharmacokinetic, pharmacodynamic and other data sets, (ii) the creation of a translation engine to integrate heterogeneous data sets into a database using the standardized ontology, and (iii) the development of statistical methods to perform data validation and outcome prediction with the integrated data. The system is designed to interface with patient electronic medical records (EMRs) in hospitals and laboratories to extract a particular patient's relevant data. The system may also be used in the context of generating phenotypic predictions and enhanced medical laboratory reports for treating clinicians. The system may also be used in the context of leveraging the huge amount of data created in medical and pharmaceutical clinical trials. The ontology and validation rules are designed to be flexible so as to accommodate a disparate set of clients. The system is also designed to be flexible so that it can change to accommodate scientific progress and remain optimally configured.

### CROSS-REFERENCES TO RELATED APPLICATIONS

This application, under 35 U.S.C. §119(e) claims the benefit of the following U.S. Provisional Patent Applications: Ser. No. 60/742,305, filed Dec. 6, 2005; Ser. No. 60/754,396, filed Dec. 29, 2005; Ser. No. 60/774,976, filed Feb. 21, 2006; Ser. No. 60/789,506, filed Apr. 4, 2006; Ser. No. 60/817,741, filed Jun. 30, 2006; Ser. No. 11/496,982, filed Jul. 31, 2006; Ser. No. 60/846,589, filed Sep. 22, 2006, Ser. No. 60/846,610, filed Sep. 22, 2006, and Ser. No. 11/603,406, filed Nov. 22, 2006; the disclosures thereof are incorporated by reference herein in their entirety.

### BACKGROUND OF THE INVENTION

1. Field of the Invention

The invention relates generally to the field of integrating data from disparate sources in different formats into a system with a standardized ontology, so that analysis can be performed on the data. Specifically, the invention is designed to enable physicians or researchers to leverage the copious amounts of genotypic, phenotypic and other medical data available, and to perform analyses on that data for medically predictive purposes.

2. Description of the Related Art

Data Sharing in Biomedicine: The Need for a Standardized Ontology and Data Validation

Clinical data is not easily reusable by disparate groups in the biomedical community because it is stored with different methods and in different formats across a wide range of information technology (IT) systems. In 2003, the NIH issued data-sharing requirements for all projects funded at or above $500K per year. The NIH requirements are intended to accelerate progress in unraveling the genome and its mechanisms by discouraging inefficiencies in collecting and recollecting similar sets of data. Roughly 40,000 studies are funded annually by the NIH, one fifth of which are subject to this requirement.

Initiatives at the Food and Drug Administration (FDA) such as the Prescription Drug User Fee Act III, combined with the exorbitant cost of drug recalls, encourage drug companies to collect clinical and genetic data to identify sound predictors of human drug responses. The fulfillment of the NIH and FDA data-sharing initiatives will necessitate a set of IT standards for the consolidation of biomedical data into a common framework.

Current Approaches to Data Integration, and Emerging Trends of Standardization

Numerous current products and research efforts offer tools that streamline data integration. These include centralized database projects exemplified by Genbank, the FMRI Data Center and the Protein Data Bank, laboratory-specific internet tools like the Flytrap interactive database, distributed data collaboration networks such as BIRN, commercial tools for data organization like Axiope, and large database systems for aggregating healthcare information such as Oracle HTB. In addition, tools have been developed to automatically validate data integrated into a common framework. Validation calls for techniques such as declarative interfaces between the ontology and the data source and Bayesian reasoning to incorporate prior expert knowledge about the reliability of each source. Bayesian analysis tools have been built to find functional associations between genetic data, such as the Multisource Association of Genes by Integration of Clusters (MAGIC).

Automated data integration and validation requires fewer human resources, but necessitates that data have well-defined a priori structure and meaning. The most successful approaches make use of a standardized master ontology that provides a framework to organize input data, as well as a technology scheme for augmenting and updating the existing ontology. This paradigm has been successfully applied in the Gene Ontology (GO), Mouse Gene Database (MGD), and the Mouse Gene Expression Database (GXD) projects, which provide a taxonomy of concepts and their attributes for annotating gene products. The Unified Medical Language System (UMLS) Metathesaurus combines multiple emerging standards to provide a standardized ontology of medical terms and their relationships. There is still much room to develop functionality that is not provided by the systems described above. There is a need for a comprehensive system which is capable of enabling researchers to i) efficiently enter heterogeneous local data into the framework of the UMLS-based ontology, ii) make necessary extensions to the standardized ontology to accommodate their local data, iii) validate the integrated data using expert rules and statistical models defined on data classes of the standardized ontology, iv) efficiently upgrade data that fails validation, and v) leverage the integrated data for clinical outcome predictions.

Predictive Tools in Cancer Treatment

Of the estimated 80,000 annual clinical trials, 2,100 are for cancer drugs. Balancing the risks and benefits for cancer therapy represents a clinical vanguard for the combined use of phenotypic and genotypic information. Although there have been great advances in chemotherapy in the past few decades, oncologists still must treat their cancer patients with primitive systemic drugs that are frequently as toxic to normal cells as to cancer cells. Thus, there is a fine line between the maximum toxic dose of chemotherapy and the therapeutic dose. Moreover, dose-limiting toxicity may be more severe in some patients than others, shifting the therapeutic window higher or lower. For example, anthracyclines used for breast cancer treatment can cause adverse cardiovascular events. Currently, all patients are treated as though at risk for cardiovascular toxicity, though if a patient could be determined to be at low-risk for heart disease, the therapeutic window could be shifted to allow for a greater dose of anthracycline therapy.

To balance the benefits and risks of chemotherapy for each patient, one must predict the side effect profile and therapeutic effectiveness of pharmaceutical interventions. Cancer therapy often fails due to inadequate adjustment for unique host and tumor genotypes. Rarely does a single aspect of a drug cause significant variation in drug response; rather, manifold idiosyncratic pharmacodynamic interactions result in unique footprint of biomolecular effects, making clinical outcome prediction difficult.

“Pharmacogenetics” is broadly defined as the way in which genetic variations affect patient response to drugs. For example, natural variations in liver enzymes affect drug metabolism. The future of cancer chemotherapy is targeted pharmaceuticals, which require understanding cancer as a disease process encompassing multiple genetic, molecular, cellular, and biochemical abnormalities. With the advent of enzyme-specific drugs, care must be taken to insure that tumors express the molecular target specifically or at higher levels than normal tissues. Interactions between tumor cells and healthy cells must be considered, as a patient's normal cells and enzymes may limit exposure of the tumor drugs or make adverse events more likely.

Bioinformatics will revolutionize cancer treatment, allowing for tailored treatment to maximize benefits and minimize adverse events. Functional markers used to predict response may be analyzed by computer algorithms. Cancer and cancer treatment are dynamic processes that can require therapy revision and combination therapy, according to a patient's side effect profile and tumor response, and potentially to genetic and phenotypic markers in the cancer. Nonetheless, having data to partially guide a physician to the most effective treatment is advantageous, and in the future, it is hoped that additional data will support efficacious decision-making at other decision nodes.

Colon Cancer as a Disease Model

The American Cancer Society estimates that 145,000 cases of colorectal cancer will be diagnosed in 2005, and 56,000 will die as a result. Colorectal cancers are assessed for grade, or cellular abnormalities, and stage, which is subcategorized into tumor size, lymph node involvement, and presence or absence of distant metastases. 95% of colorectal cancers are adenocarcinomas that develop from genetically-mutant epithelial cells lining the lumen of the colon. In 80-90% of cases, surgery alone is the standard of care, but the presence of metastases calls for chemotherapy. One of many first-line treatments for metastatic colorectal cancer is a regimen of 5-fluorouracil, leucovorin, and irinotecan.

Irinotecan is a camptothecin analogue that inhibits topoisomerase, which untangles super-coiled DNA to allow DNA replication to proceed in mitotic cells, and sensitizes cells to apoptosis. Irinotecan does not have a defined role in a biological pathway, so clinical outcomes are difficult to predict. Dose-limiting toxicity includes severe (Grade III-IV) diarrhea and myelosuppression, both of which require immediate medical attention. Irinotecan is metabolized by uridine diphosphate glucuronosyl-transferase isoform 1a1 (UGT1A1) to an active metabolite, SN-38. Polymorphisms in UGT1A1 are correlated with severity of GI and bone marrow side effects.

Prior Art

In U.S. Pat. No. 5,824,467 Mascarenhas describes a method to predict drug responsiveness by establishing a biochemical profile for patients and measuring responsiveness in members of the test cohort, and then individually testing the parameters of the patients' biochemical profile to find correlations with the measures of drug responsiveness. In U.S. Pat. No. 7,058,616 Larder et al. describe a method for using a neural network to predict the resistance of a disease to a therapeutic agent. In U.S. Pat. No. 6,958,211 Vingerhoets et al. describe a method wherein the integrase genotype of a given HIV strain is simply compared to a known database of HIV integrase genotype with associated phenotypes to find a matching genotype. In U.S. Pat. No. 7,058,517 Denton et al. describe a method wherein an individual's haplotypes are compared to a known database of haplotypes in the general population to predict clinical response to a treatment. In U.S. Pat. No. 7,035,739 Schadt at al. describe a method is described wherein a genetic marker map is constructed and the individual genes and traits are analyzed to give a gene-trait locus data, which are then clustered as a way to identify genetically interacting pathways, which are validated using multivariate analysis. In U.S. Pat. No. 6,025,128 Veltri et al. describe a method involving the use of a neural network utilizing a collection of biomarkers as parameters to evaluate risk of prostate cancer recurrence. In U.S. Pat. No. 6,489,135 Parrott et al. provide methods for determining various biological characteristics of in vitro fertilized embryos, including overall embryo health, implantability, and increased likelihood of developing successfully to term by analyzing media specimens of in vitro fertilization cultures for levels of bioactive lipids in order to determine these characteristics. In U.S. Patent Application 20040033596 Threadgill et al. describe a method for preparing homozygous cellular libraries useful for in vitro phenotyping and gene mapping involving site-specific mitotic recombination in a plurality of isolated parent cells. In U.S. Pat. No. 5,994,148 Stewart et al. describe a method of determining the probability of an in vitro fertilization (IVF) being successful by measuring Relaxin directly in the serum or indirectly by culturing granulosa lutein cells extracted from the patient as part of an IVF/ET procedure. In U.S. Pat. No. 5,635,366 Cooke et al. provide a method for predicting the outcome of IVF by determining the level of 11.beta.-hydroxysteroid dehydrogenase (11.beta.-HSD) in a biological sample from a female patient. In US Patent application 20060052945, Rabinowitz at al. describe a system for integrating and validating medical data into a standardized database.

### SUMMARY

The system described herein enables clinicians and researchers to use aggregated genetic and phenotypic data from clinical trials and treatment records to make the safest, most effective treatment decisions for each patient. Modern information technology allows research institutions, hospitals and diagnostic laboratories to accumulate valuable medical data. Currently, data collected at each institution tends to be independent in format and ontology, making it difficult to combine or compare data from disparate sources. There is a burgeoning need to integrate and interpret medically-relevant genetic and phenotypic data to enable clinicians to make better treatment decisions, faster, based on sound predictors of medical outcome.

In one aspect of the invention, a system is described to facilitate the standardization of a wealth of information that lies in a huge number of electronic and paper medical record systems around the globe. While the information lies in difficult to access, often proprietary, heterogeneous data storage systems, it remains underutilized. The system described herein lowers the barrier to the aggregation of large sets of data in a format that is accessible to meta-analysis and other data mining techniques. The system is also designed to be flexible, so that it can change to accommodate scientific progress and remain optimally configured.

One aspect of the invention involves the creation of standardized ontologies for genetic, phenotypic, clinical, pharmacokinetic, pharmacodynamic and other types of medically related data sets. The ontology is designed to be flexible to allow for the incorporation of data sets and data types that may not be foreseen at the outset. This flexibility can accommodate for the advance of medicine and science, where new topics, and the significance of new independent variables are recognized. It can also accommodate for the incorporation of independent variables that may not yet be recognized to be important, but whose significance may not yet been discovered. In addition the flexibility can also accommodate for the fact that the creators of an ontology can not a priori fully understand all aspects of medicine.

One aspect of the invention involves the creation of a translation engine which is capable of integrating heterogeneous data sets into the standardized ontology. There are a multitude of ways in which medical data can be measured and stored, including but not limited to differing storage media, database designs, study parameters, sets of measured variables, data formats, and the various combinations thereof. Additionally, each medical system that stores data may have different protocols and formats for accessing data. In order to integrate such disparate sets of data, the system described herein uses a method that greatly facilitates the translation of this data into a unified format that can be accessed and universally understood. As part of the system design, it is recognized that the easier it is to use and the more automated the system is, the lower the barrier will be for entities to contribute data to the aggregated database, thus enhancing its value to the medical community.

The system is designed to interface with patient electronic medical records (EMRs) in hospitals and laboratories to extract a particular patient's relevant data. The system may also be used in the context of generating phenotypic predictions and enhanced medical laboratory reports for treating clinicians. The system may also be used in the context of leveraging the huge amount of data created in medical and pharmaceutical trials. The ontologies are designed to be flexible so as to accommodate a disparate set of clients. The system disclosed herein can be used for individual files, for groups of files and for entire databases of medical data. The system can be used in the context of a single or small group patients, a single or group of doctors, a single or group of medical studies or trials, a single or group of medical practices, a single or group of hospitals, or any other set of medical records. Once the appropriate translation cartridge has been created, all data available in a given format can be translated and aggregated into a system using a standardized ontology.

In another embodiment of the invention the system is extended to streamline the integration of other data types, including pharmacodynamic (PD) and locally defined classes of data, especially those found in clinical trials. The ontology and method for validation are expanded to accommodate cartridge creation by a pharmaceutical company for their own clinical trial data, enabling integration into computable format from multiple laboratories. This same system can also be used by diagnostic testing companies who want to offer an efficient data analysis service to the hospital laboratories that use those tests. Although the system described elsewhere is a generic system for use by multiple diagnostic testing and pharmaceutical companies, it is important to note that the cartridge generation engine can be designed to meet the needs of major pharmaceutical companies such as Pfizer Inc. and diagnostic testing companies such as Genzyme.

Another aspect of the invention is to check, or validate the data that has been integrated into a database from external sources. There are many potential sources of error in the integration of data initially stored in diverse record systems. As the validity of the underlying data is critical to any predictive efforts, an important part of any system designed aggregate data is to ensure its fidelity, and to identify, as much as possible, any data that is in error. It is impossible to correct every error with 100% certainty, but the types of errors which introduce the largest inaccuracies in subsequent predictions, those that fall significantly outside the norms, are also the ones that are easiest to identify. The use of expert rules and expectations, in combination with statistical methods can result in a significant reduction in the number of data errors, and thus an increase in the accuracy of the analyses based on the data.

Another aspect of this invention involves the use of the aggregated data to make better phenotypic, clinical and medical predictions. With a large amount of genotypipc, phenotypic and medically related data on hand, mono- and multifactorial correlations not previously recognized can be discovered. Once the system described herein has integrated large amounts of data into a database with a standardized structure and format, it becomes feasible to run analyses and meta-analyses in situations where previously the smaller quantity of data points would have resulted in a lack of statistical significance, or a lack of recognition of variable correlation due to insufficient quantities of patients of a given sub-category.

Certain embodiments of the technology disclosed herein describe a system for making accurate predictions of phenotypic outcomes or phenotype susceptibilities for an individual given a set of genetic, phenotypic and or clinical information for the individual. In one aspect, a technique for building linear and nonlinear regression models that can predict phenotype accurately when there are many potential predictors compared to the number of measured outcomes, as is typical of genetic data, is disclosed. In certain examples, the models are trained using convex optimization techniques to perform continuous subset selection of predictors so that one is guaranteed to find the globally optimal parameters for a particular set of data. This feature is particularly advantageous when the model may be complex and may contain many potential predictors such as genetic mutations or gene expression levels. Furthermore, in some examples convex optimization techniques may be used to make the models sparse so that they explain the data in a simple way. This feature enables the trained models to generalize accurately even when the number of potential predictors in the model is large compared to the number of measured outcomes in the training data.

In another aspect, a phenotypic or clinical outcomes can be predicted using a technique for creating models based on contingency tables that can be constructed from data that is available through publications such as through the OMIM (Online Mendelian Inheritance in Man) database and using data that is available through the HapMap project and other aspects of the human genome project is provided. Certain embodiments of this technique use emerging public data about the association between genes and about association between genes and diseases in order to improve the predictive accuracy of models.

In another aspect of the invention, the predictions that are made based on the aggregated data can be used to generate enhanced reports with the purpose of organizing the data and analyses in a way that is most useful to physicians or clinicians, and most beneficial to patients. In some cases this report may give details about the most appropriate course of treatment for a given patient with a given illness. In some cases this report may recommend personalized preventative measures in an effort to avoid phenotypes or conditions for which the individual is predisposed.

In another aspect of the invention, the aggregation and validation of data can be done in an academic context. This could done be for the purpose of building academic research databases, such as PharmGKB, or other academic data repositories designed to facilitate medical research. In another aspect, the aggregation and validation of data may be done in other contexts, such as pharmaceutical development.

### TABLE OF FIGURES AND CHARTS

FIG. 1. Excerpt of ontology.

FIG. 2. Data entry spreadsheet.

FIG. 3. A segment of the CSO Describing a drug administration event.

FIG. 4. System computer code extract.

FIG. 5. System computer code extract.

FIG. 6. Information about SNP, Patient sample and Affymetrix Genotyping Arrays represented in GMA CSO

FIG. 7. Add Element page in cartridge generation web interface.

FIG. 8. Sample preview report in cartridge generation web interface.

FIG. 9. The interface architecture.

FIG. 10. A segment of the pharmacokinetics ontology, addressing the high-level element drug dosing event.

FIG. 11. Process of translation with a cartridge.

FIG. 12. XForms Generated Cartridge

FIG. 13. XSL Transform using Altova MapForce

FIG. 14. Decision flow diagram for selection of data classes with associated XSD schema.

FIG. 15. Physical layout of enhanced reporting system.

FIG. 16. Architectural overview of the enhanced reporting system.

FIG. 17. Example of data outside of expected bounds.

FIG. 18. Data validation.

FIG. 19. Data (re)submission process.

FIG. 20. Schema describing how system internally translates and store bulk data from raw measurement files, and provides external interfaces to retrieve data in well understood formats.

FIG. 21. The components of the system

FIG. 22. Screenshot of Mantis bug tracking system for PharmGKB project.

FIG. 23. Login screen.

FIG. 24. Welcome screen.

FIG. 25. Cartridge selection and spreadsheet generation page.

FIG. 26. Create cartridge page.

FIG. 27. Drug dosing event page.

FIG. 28. Add description element page.

FIG. 29. More information page.

FIG. 30. Error warnings page.

FIG. 31. Data integration.

FIG. 32. Sample My Datasets webpage.

FIG. 33. Sample element from cartridges page.

FIG. 34. Sample window.

FIG. 35. Sample spreadsheet.

FIG. 36. Sample datasets list.

FIG. 37. Validation running window.

FIG. 38. Review errors button.

FIG. 39. List of records with warning flags.

FIG. 40. Sample record in need of validation.

FIG. 41. Example of error overridden message.

FIG. 42. Example of record removal message.

FIG. 43. List view of validated records within a dataset.

FIG. 44. Example of validated data message.

FIG. 45. DataSets tab shows all submitted data, submission date, and results of validation, and allows the user to view delete, or correct records.

FIG. 46. Cartridges tab allows user to create Excel spreadsheets for data entry, delete or copy and modify a previously-created cartridge.

FIG. 47. User specification of Irinotecan drug dosing event during cartridge creation.

FIG. 48. ANC Prediction, given UGT1A1 SNPs and Irinotecan metabolite measures.

FIG. 49. Mock enhanced report for colon cancer.

### DETAILED DESCRIPTION

Modern information technology allows research institutions, hospitals and diagnostic laboratories to accumulate valuable medical data. Currently, data collected at each institution tends to be independent in format and ontology (when an ontology exists), making it difficult to combine or compare data from disparate sources. There is a burgeoning need to integrate and interpret medically-relevant genetic and phenotypic data to enable clinicians to make better treatment decisions, faster, based on sound predictors of medical outcome. The focus of this system is creating a product for pharmaceutical companies, diagnostic testing companies, hospital laboratories using diagnostic tests, and clinicians making difficult treatment decisions that could be guided by distillation of available medical data.

This software system has five main aspects, which may be used separately or in combination with other aspects. The first aspect involves defining and creating a standardized ontology that can accommodate all of the relevant data subsets. In some cases, relevant data classes may not have been specifically designed into the ontology, but the ontology is designed to be flexible and allows for the definition and creation of as many new data classes as are needed.

The second aspect involves integrating data from disparate sources into the standardized ontology. In order to do this, an interface based on the standard ontology is generated that allows a researcher or other agent to describe their data fields appropriately. Following this, the system generates a translation definition called a “cartridge” that is capable of assimilating the data from the input data of the researcher or agent into the appropriate locations of a database using the standardized ontology, or to create new locations where appropriate. Finally the data is integrated.

The third aspect involves validating the data, ensuring that spurious or incorrect data that could skew later analyses is not integrated. In order to do this, a set of relationships between the standardized data classes is determined that describes expected limits and/or patterns of the assimilated data based on statistical models and/or expert rules. Then the likelihood of the validity of the assimilated data is determined based on those limits and rules. Data that do not conform to the expectations are flagged for review by a knowledgeable person.

The fourth aspect involves using statistical techniques operating on the aggregated data to make phenotypic, clinical or other predictions involving an individual, or group of individuals. The method uses mathematical modeling techniques that operate on relevant aggregated medical data from germane patient subpopulations to make the best predictions possible. The models may be linear or non-linear, and they may be based contingency tables.

The fifth aspect involves the creation of an enhanced report that can present the features of the analysis that are most relevant to the agent treating the individual(s) in question. For example, if a physician is treating a cancer patient, the report may contain information concerning the particular mutations present in the cancer, possible treatment options, and the likely outcomes of each of the treatments given the particular characteristics of the patient and the cancer in question.

Creating a Context Specific Ontology

The first step in aggregating data into a unified format is to design a system of organization that is detailed and flexible enough to accommodate all possibilities data and data classes, as well as the relationships between those data. The crux of describing data is the act of linking up concepts with a context specific ontology (CSO), which relates “concept unique identifiers” (CUIs) to each other in a specific way. For example, one can only derive meaningful data from a metabolite measurement when one describes the context in which that measurement was collected, such as the original drug dose, dosing schedule, and measurement time points. The CSO enforces collection of all contextual data to ensure that aggregated data is unambiguous.

A key goal of the invention is to support sharing between the greatest number of researchers and information systems. Consequently, it is crucial that all data submitted to the standardized ontology be unambiguously defined. The National Library of Medicine has created a knowledge source, the Unified Medical Language System (UMLS) Metathesaurus, which relates data classes from over 100 controlled vocabularies and classifications, including the Systematized Nomenclature of Medicine Clinical Terms (SNOMED-CT), Medical Subject Headings (MeSH), Logical Observation Identifiers Names and Codes (LOINC), and RxNorm. The UMLS Metathesaurus preserves the concepts, hierarchical contexts, and inter-term relationships present in its source vocabularies. In one embodiment of the invention, the definitions used in the CSO are based on these systems.

Despite the extent of the UMLS ontology, it is often not detailed enough to accommodate all local data. One embodiment involves an approach to extending the ontology. Although ontology standards exist which allow arbitrary extensions and combinations of concepts into necessary higher order concepts, allowing users such latitude can be unwieldy. It is most effective to constrain the space of possible concepts to a level which meets the following guidelines:

1) Maximize commonality across researchers by constraining definition latitude for researchers.

2) Provide common templates for common concepts.

3) Allow extensions when common concepts do not suffice.

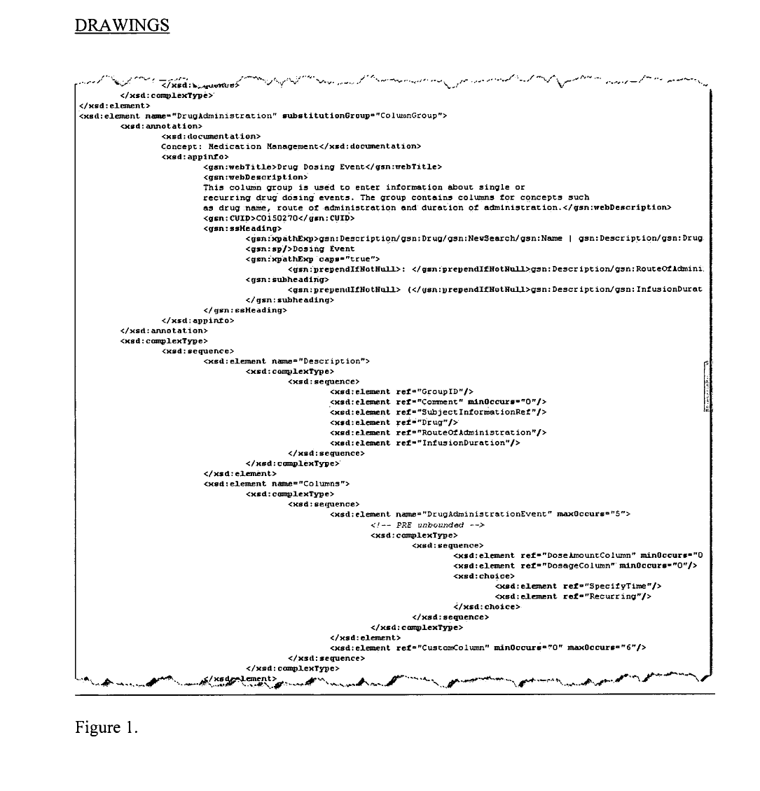

4) Ensure practicality by encapsulating knowledge one domain at a time. By following these guidelines, a Context Specific Ontology (CSO) has been developed which builds high level concepts out of atoms defined by UMLS, HL7, and de facto PharmGKB standards. Many leaf elements of the CSO are associated with UMLS Concept Unique Identifiers (CUIs) that define the meaning of the associated data class. An excerpt from the ontology is shown in FIG. 1.

In order to completely define researchers' data sets, concepts also need to be associated with units of measure. Instead of redefining lists of units, the CSO leverages measurement units adopted by the HL7 standards body. The standard list of units used in medical tests can be surprisingly large and varied depending on the use case. HL7 has been attempting to normalize this list via the UCUM (Unified Code for Units of Measure). UCUM, however, is at the wrong level of granularity (too detailed) to be of much use in practice. There is an effort to include support for the UCUM standard in the next version of ELINCS, an HL7 messaging specification (sponsored by the California Healthcare Foundation) with the goal of standardizing the electronic reporting of test results from clinical laboratories to electronic health record (EHR) systems. As a part of this effort, to ultimately incorporate UCUM in ELINCS, researchers have developed a list of a set of commonly used UCUM codes for units in healthcare.

In the user interface, the system splits unit lists to common and full lists to streamline usability. The UCUM standard also provides a conversion table to allow the system to scale between associated units for meta-analysis purposes. The integrity of data is initially validated by means of the high-level formatting information encoded in the pharmacokinetics XSD schema. The low level format is then validated based on the HL7 format information in the meta-database. Properly formatted data is integrated into the standardized ontology to be validated more thoroughly by means of expert rules and statistical models.

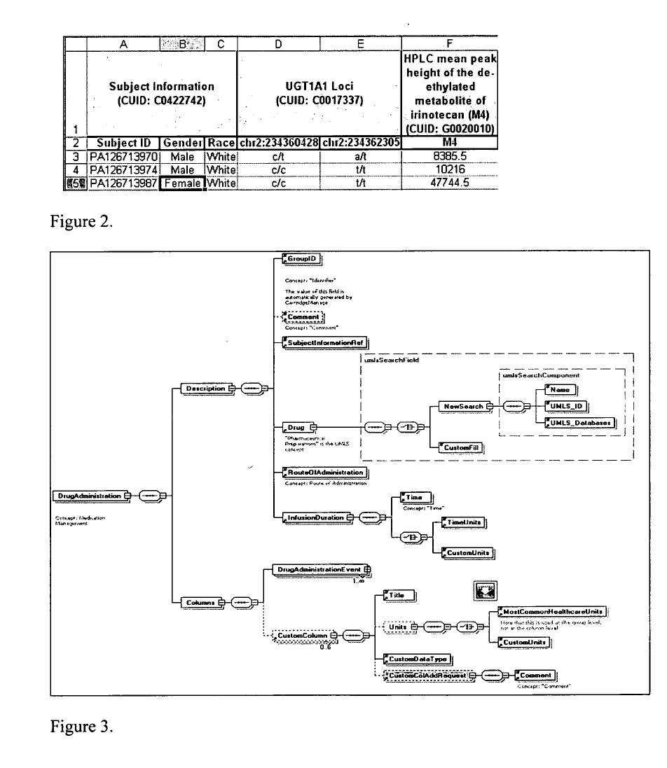

In one embodiment, the context necessary for understanding the data is provided in a segment of XML that is compliant with the CSO, and describes the set of concepts that occur together, the relations between those concepts, and the data format to fully describe the data submitted in each column of the Excel spreadsheet. Each segment of XML describing a column of data is associated with a unique system ID. From this XML, a group heading with UMLS concept IDs and column headings for each data element is created, as illustrated in FIG. 2.

In one embodiment, when data is submitted, it may have context-specific formatting requirements, including logical groupings of data classes and required fields. This information is contained in a Context-Specific Ontology (CSO) that is rendered as an XML Schema Definition document (XSD). In one example, the pharmacokinetics XSD specifies a data format for capturing information about how drugs are applied to and metabolized by subjects. This XSD document defines elements that characterize a set of events, ranging from the administration protocol of drug doses to the measurement of drug metabolites in different body compartments. A user interface is automatically generated based on the CSO, which guides the user through selecting relevant data classes and entering meta-data for the dataset they are submitting. This process outputs a segment of XML which is compliant with the CSO XSD and which describes the meaning, format and context of each piece of data submitted to the system. This makes the data truly computable. The CSO for all integrated data can be disseminated from a recognized authority, for example the company that owns the rights to the patent covering the disclosed system. A link on the group and column headings of data published by the authority connects to the authority and provides information on the meaning, format and context of the model using the user interface that is used in creating the cartridges, as described below.

Overview of the Organization and Function of the CSO

In one embodiment of the invention, the CSO is organized as follows (see FIG. 3): A cartridge, which is the root element of the CSO, must contain one or more “column groups” and each column group must contain at least one “description field”—which provides metadata that refines the context of the column group. Each column group also contains at least one “column field” which describes a particular column or data class that resides within the column group. The description fields for the column group provide context for the column fields that belong to that column group. The Excel spreadsheets that are generated from cartridges have two rows of headings. The top row of headings corresponds to the column groups in the CSO and is created based on the description fields. The second row of headings corresponds to the individual columns and is created based on the column fields.

An example of a column group is “Drug dosing event,” and an example of a top-level heading for the column group is, “[C0123931] Irinotecan: MSH; Dosing Event: Intravenous Infusion (90 minutes) (CUID: C0150270).”—Note that the drug is identified with its UMLS CUI allowing this data to be correlated with other pharmacogenomic data where Irinotecan was administered as a 90 minute intravenous infusion. The description fields corresponding to this column group include “drug name,” “route of administration,” and “infusion duration.” Example column fields belonging to the cartridge group are “Dose amount (mg): (CUID: C0870450)” and “Dosage (mg/m2): (CUID: C0870450).” These fields provide further details about the intravenous infusion of irinotecan. Both description fields and column fields can be defined as either necessary or optional, and the maximum and minimum times an element can occur can be restricted in order to make the cartridge more or less flexible.

In one embodiment, the ontology contains the following high level elements or column groups: Subject Information, Human Gene Locus, Drug Dosing Event, Concentration Test, Clearance Test, Volume of Distribution Test, Area under the Curve Test, Half Life Test, Custom Laboratory Test and Custom Column Group.

All of these elements are defined in the CSO, which is expressed in the form of an XML Schema Definition (XSD) that defines valid elements in the cartridge. XSD is a widely used language for defining what constitutes a valid XML documents within a specific domain. The CSO is designed so that it can be parsed by the system to generate web forms that users can use to create cartridges conforming to the restrictions and definitions contained in the CSO. In addition to the standard XSD tags, the system uses a specialized tags for generating column headings and defining the data types of the columns of the cartridge (“Text,” “Number,” or “Date”). Other specialized tags are used to add human-readable documentation to the cartridge creation forms. For example, the human-readable description of Drug Dosing Event is: “This column group is used to enter information about single or recurring drug dosing events. The group contains columns for concepts such as drug name, route of administration and duration of administration.”

FIG. 3 illustrates a segment of the XSD that describes a Drug Administration which constitutes part of a Drug Dosing Event. Each series in the schema [symbol] involves a series of data class selections by the researcher, every choice in the schema [symbol] involves selecting elements from a pull-down menu, and every leaf element [symbol] involves either meta-data entry or selection from a pull-down menu. Attributes associated with each data class in the schema describe whether the data element is used to refine the headings of the Excel template, to define one of the columns in the template, or simply to guide the class selection process.

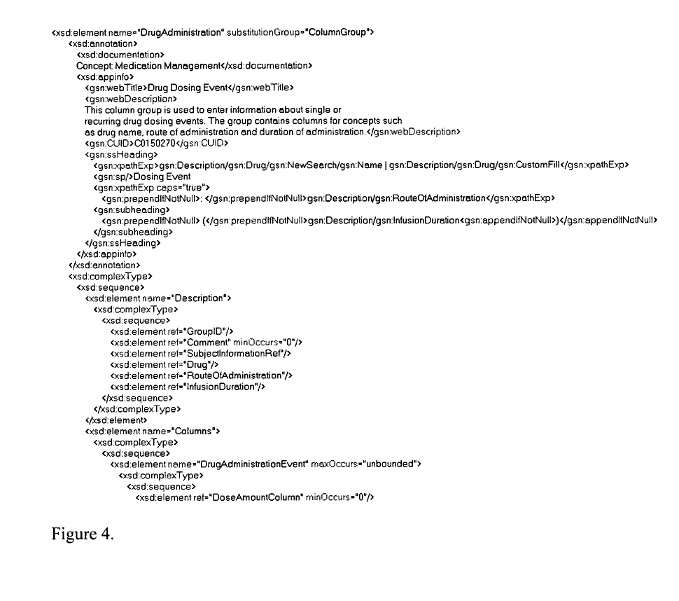

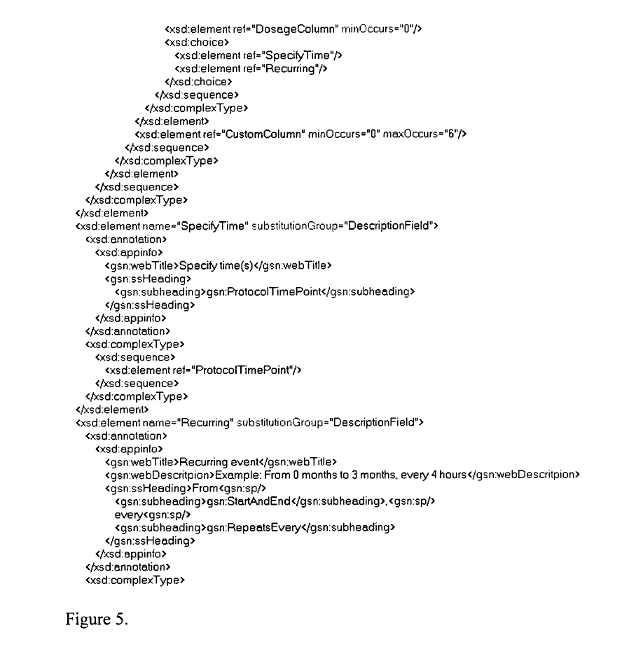

FIGS. 4 and 5 show two screenshots of the XSD code for the Context Specific Ontology for Pharmacokinetics. Code is omitted that would be obvious to one skilled in the art. For this illustration, it is assumed that the user of the template is proficient in XSD and XML computer languages.

Creating a CSO in the Context of Genotyping Data

In another embodiment of the invention, a method is specified to generate a standardized format for capturing and rendering high throughput genotyping data. This is referred to as the Genotyping MicroArray CSO, or GMA CSO. Many types of data can be integrated into a standardized ontology. The following description will focus on genetic data.

Genotyping arrays provide the ability to measure multiple SNPs on an individual's genome. For accurate interpretation of this large amount of data several things must be known: the position of these SNPs on the chromosome, the alternative configurations (alleles), how frequently they are seen in particular ethnic populations, and also need the disease or pharmacogenomic phenotypes that are associated with particular SNPs.

Genotyping arrays from can provide a measurement for the presence (or absence) of a particular nucleotide at thousands of these SNPs. In addition to mapping the measurement from the measuring device to a particular SNP position on the chromosome, it is important to capture the relevant meta-data about that particular SNP from public sources such as dbSNP. It is also important to know the experimental conditions under which the DNA is isolated, and the experiment design. This meta-data will be incorporated into GMA CSO.

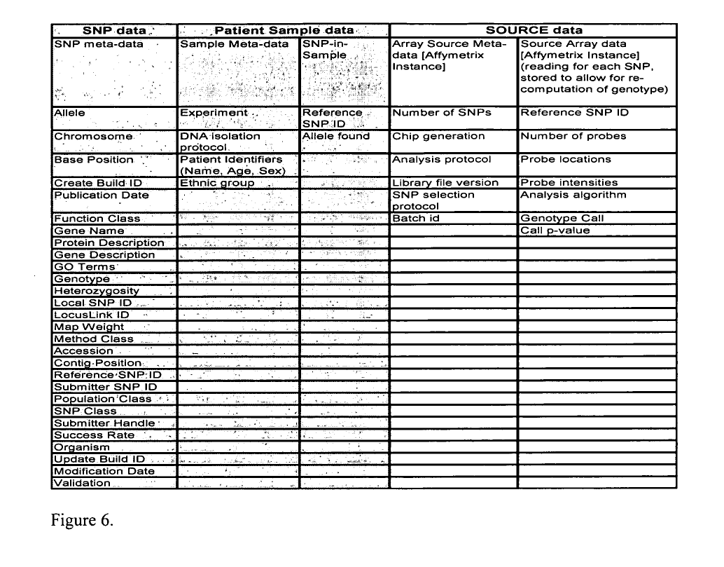

A lot of information such as allele frequencies, population distribution, gene-association and disease-association is available about each SNP in the public domain from resources such as dbSNP and PharmGKB. Relevant elements from the xsd's of both these sources may be represented in GMA CSO. For example, both dbSNP and PharmGKB contain elements to represent the chromosome location, base position and the allele information for a SNP. dbSNP provides the population in which the SNP was observed and the frequency with which alleles were observed. PharmGKB contains additional information about the SNP's role in drug-metabolism. PharmGKB provides the pharmacological significance of the SNP (if any) by means of the <gene> element which links SNPs to pharmacological information via the <namedAlleles>, <polymorphismXref> and the <pharmacogenomic Significance> elements. For a complete list of data items to be represented by GMA CSO (see FIG. 6).

Scanning the Genotyping arrays generates data about the intensity values from each probe on the chip, which is interpreted by the GCOS software using the Dynamic Model Mapping algorithm (DMPA) to generate a call and a p-value for the presence of a particular allele in the probed DNA. The GCOS software summarizes the intensity readings from 40 probes for each SNP. Because the DMPA interpretation can change, and because one goal may be to estimate the probability of a correct call on the SNP, it is important to capture the underlying probe intensity data and the probe layout for each SNP along with the result output by GCOS.

Each probe on the some genotyping arrays, such as the Affymetrix 100K and 500k Genotyping arrays is linked to a known SNP and identified by a RefSNP id from dbSNP. This is crucial to relating observed SNP's in an individual with the known role of a particular SNP in causing disease (derived from PharmGKB or OMIM) and this will be captured in GMA CSO.

In one embodiment, genotype data from an individual may be captured in an XML document that conforms to the GMA CSO and contains values for elements capturing SNP information, array information and links between SNP and Array elements. It is possible to develop an all encompassing standard, such as the MAGE-OM, for capturing all the possible ways in which a genotyping array (or other genotyping technologies) can be used. However it is sufficient to use a GMA CSO that is a subset of whatever standard is eventually formed, possibly derived from MIAME and MAGE-OM. The XML data document may be generated using the same approach that has been described elsewhere in this document to support data submissions to pharmGKB. The translation engine will create an XForms user interface, based on GMA CSO, with which the user can select data classes relevant to their local data, enter relevant meta-data, and select the genotyping array output files in which the genotyping array data is captured. The system will then generate an Excel spreadsheet template in which patient-specific information can be entered, together with a cartridge for validating and integrating the information into the standardized format. It may also be useful to develop a JAVA plugin that enables the cartridge to integrate individual genotype data into the GMA CSO ontology.

In one embodiment, the GMA CSO may be applicable to data from all gene micro arrays, and not be bound to a single vendor. However, it is necessary that source data is not lost so that SNP inferences can be re-calculated from original data in case of method improvements in the future. To that end, the schema may have a Source data section, which would include original data from each chip. Source data will be tailored for each chip, and will require knowledge of the chip vendor itself for interpretation. Note that some of the information in SNP data column will also be covered by the Affymetrix “library” files that link particular probe sets to SNPs in the genome, and also that the GMA CSO may also include complete copies of SNP meta data, or references to dbSNP entries.

Creation of a User-Friendly Web Interface: Functional Overview

The most labor intensive aspect of the invention is expected to be the need for a user to describe the data fields in a local database appropriately, such that the data can be integrated into a standardized format. Since there are a large variety of medically oriented databases, some of which are proprietary systems, some of which are legacy systems with unusual formats, and most of which are idiosyncratic in some way, in order to leverage the data in these systems it is necessary for significant human interaction in drawing the appropriate connections in defining the data. As such, it is important that a method is used that is efficient and easy for the user. The process begins with a user who is uploading medically relevant data, such as clinical outcome data. He first needs to describe his research outcome data in terms of a Context Specific Ontology (CSO).

In one embodiment, through a web interface, the user chooses the data classes which represent the column groups, and individual columns of the table of result data, and fills in necessary parameters to fully describe his data. For example, if a column in his data spreadsheet records a drug dosage given to a patient, the researcher describes the units of measurement of the dosage, the drug name (using UMLS) and the method of dosage (oral, intravenous, etc . . . ) to fully describe the dosing event. The system enforces the CSO's constraints to force the researcher to fully describe his data. After he describes each column in his data set he saves the description as a cartridge. All the details that the system collected from the researcher are stored in a structure called a “cartridge”. The cartridge now fully describes his data in a way that it can be understood by the standardized ontology.

The user (or any other user) can download an Excel spreadsheet template for his (or any) cartridge. The spreadsheet template columns align with the cartridge's column descriptions. The user enters or cuts-and-pastes the data into the template and can now upload the data for validation and storage. This template can be reused over and over again by this user or any user wishing to upload data in a similar format. Once uploaded to the system servers, the system validates the structure of the spreadsheet according to the following simple checks:

1) Is there the correct number of column groups?

2) Is there the correct number of columns per group?

3) Does each column group have its expected name?

4) Does each column have its expected name?

If these initial checks pass then the system loads the data into its internal representation as described by the cartridge. The records are all uploaded from the spreadsheet into the system's database. The user then can “validate” the new data.

Cartridge Generation

In another embodiment of the invention, the user can build a translator, or “cartridge” to translate his local data into a CSO compliant dataset. The local (or source) is often stored in the dreadsheet, but may be stored in a database, or in XML, or in an EMR or any other storage medium. To build a cartridge the user can select a CSO from a drop down list of active ontologies which is appropriate for his domain of data (e.g. pharmacokinetics). The user will then enter the name of the new cartridge and click the submit button. This takes user to a page where the cartridge is built (see FIG. 7). The user will select from the list of high level elements on the left (these are the highest level elements of the CSO). An example of a high level element is Drug Dosing Event, Metabolite Measurement Event, etc. The user knows what data he has and uses this page to select the matching high level elements that match their data set. He selects the high level elements from the list on the left and is then taken to a detailed web form at which he can select/specify the data classes for each high-level element. Once the user has gone through this process for each high-level element, the element is displayed on the right along with a display name so that the user can keep track. The element on the right can be deleted, edited, or moved up/down relative to other elements. Moving up and down will change the order of the associated columns in the spreadsheet.

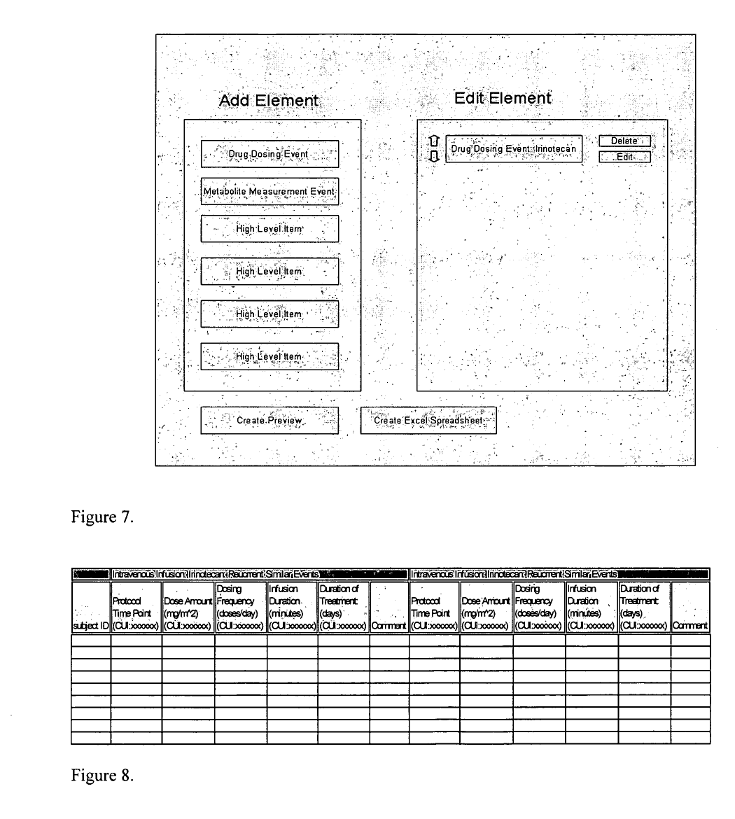

The user can preview what the data entry template looks like by selecting the Preview button. This preview is in the form of an HTML page. The preview shows the selected high level items, and low level classes, with formatted group headings and column headings, each associated with the relevant CUIs. The user can then make changes in selections and rerun the preview report. An example of the preview report is given in FIG. 8. Once the user has run a preview report the actual cartridge can be created. The user does this by clicking the “Create Excel Spreadsheet Button”. The user can then save the Excel Spreadsheet.

In one embodiment of the invention, the system may contain any number of account administration features that are common in computer based multi-user systems. These features may include but are not limited to the following examples. One page may allow system administrator to edit the users. There may be a link on the Organization line to a page where a new Organization can be created. There may be a page that will allow user to add an organization to the list of organizations in the system. Each organization may be associated with certain fields such as user groups or profiles. Certain users may only be allowed to view data, while others may submit/edit and delete data. Other user may be able to edit, add users and perform administrative functions on the system. The navigation bar may only display the tasks/pages that a user has access to. The administrative user may have all pages in the navigation bar, while the view data user may have a limited set of pages. The system may have three levels of users: system administrator, privileged user, and standard user. There may be a Reset Password Page that is used when a user has forgotten password and received a temporary password via email. The user may be returned to the login page and after successful login is routed to this page to reset password. There may be a Login Page that is the starting point for the system. This page may allow the user to login to system, take action to retrieve forgotten password, take action to edit profile. The login may have a field for user name and password. A submit button may also be displayed. A forgotten password link may enable a user to enter email address and have a temporary password sent to email account. The users may use this temporary password but will be routed to change password screen on first login.

Functional Specification of Cartridge Generation

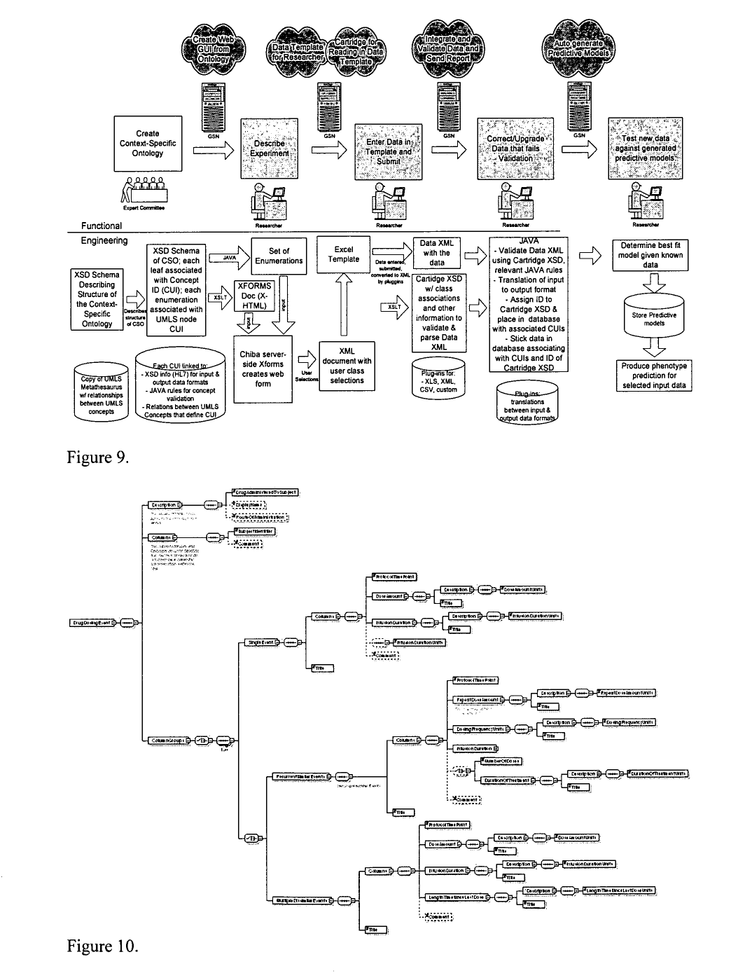

In one embodiment of the invention, is illustrated in FIG. 9 illustrates the functional specification (above dotted line) and the engineering specification (below dotted line) for the system workflow. The functional specifications are described first, followed by a description of how each functional component ties to the engineering specification. The engineering blocks (roughly) are arranged below the corresponding functions.

In one embodiment, the process begins with a team of experts creating a context-specific ontology (CSO) which contains all the data classes and context-specific formatting requirements, including groupings of data classes and required fields. For example, a pharmacokinetics CSO may specify a data format for capturing information about how drugs are applied to and metabolized by subjects, in order to support pharmacokinetic data associated with a particular indication. All functionality automatically provided by the system authority is in shown in grey clouds; all the user interaction with the system is shown in grey rectangles.

From the CSO, a server-side web interface is generated that guides the researcher through a series of data class selections, mostly from pull-down menus, in order to accommodate the user's local data. When prompted for the type of data to be added, if the researcher selects a pharmacokinetic data type (e.g. drug dosing event or metabolite measurement event), the resulting information will be integrated with a cartridge. If the researcher enters a non-pharmacokinetic data type, the researcher will be prompted to enter a descriptive name and definition for the data class, and the data will be stored outside of the standardized ontology.

Once the researcher's selections are made, the system automatically generates an Excel spreadsheet template with group headings that provide context for related data classes, and column headings that include the concept CUIs. The system may also generate a cartridge that validates the formats and values of data submitted using the template, and that integrates the data into the standardized ontology. The user then pastes relevant data into the template, selects the relevant cartridge, and submits their data for validation and integration.

Programming Specifications of Cartridge Generation

One embodiment of the invention is illustrated in FIG. 10, where a segment of the pharmacokinetics ontology, addressing the high-level element Drug Dosing Event is shown. Each leaf in the pharmacokinetics ontology may be associated with a CUI. In addition, certain points in the ontology that require enumerations (e.g. drug names) that will be associated with a CUI from UMLS so that the appropriate list of alternatives can be generated by querying a copy of the UMLS metathesaurus. The format of the database tables will be a flex schema.

The web interface used to select/specify data classes may be implemented using Chiba server-side Xforms. XSLT will be used to translate the CSO into an XForms documents implemented as X-HTML. Also, Java code may be used to expand all enumerations in the CSO into a list by querying the UMLS Metathesaurus database. The lists may be stored in separate files and will be hyper-linked into the XForms document. The XForms, in creating the web interface, may pull the enumerations from the file created by the JAVA code.

Once the user has stepped through their selection of data classes, the system will generate a cartridge that contains all of the user's data class selections. This cartridge is then used to generate the Excel spreadsheet template. The cartridge contains all of the class associations and other information to validate and parse the information that is submitted according to the Excel spreadsheet template.

The user inputs data into the spreadsheet, selects the relevant cartridge and submits the data. The system converts the Excel template into an XML document. The system will use plug-ins to convert certain incoming data formats (e.g. a list of amino acids for the RT enzyme) to outgoing data formats (e.g. mutation list for RT enzyme). Once all data has been converted into the correct format, the data will be stored in the database in CUI-value pairs that are also associated with the ID for the cartridge. This data is saved in the database as a document. The cartridge is also stored in the system for future use.

Augmentation of the Standardized Ontology

To enable efficient extension of the ontology, in one embodiment, users will be enabled to use the cartridge generation engine to electronically submit additions to the standardized ontology. Augmentation of the ontology will be implemented through a web interface in which the user will be able to add and define a data class in the course of designing a cartridge through a “custom columns” option. The user will be prompted for a set of information required to define that data type, such as units and UMLS concept searches for what's being measured and the measurement procedures. By encouraging researchers to submit additional descriptive meta-data when they add their own data class, the process by which the context-specific ontology can be augmented to facilitate creation of data-specific cartridges will be streamlined.

The system is created around an architecture guided by PharmGKB's pharmacokinetic data, but is extended to accommodate additional data classes, including pharmacodynamic and genomic data. The cartridge generation engine is productized so that cartridges can be generated to specifically meet the data integration needs of pharmaceutical companies, biotechnology companies, researchers and whomever else may use it. Additional validation rules can be generated based on the user's data requirements.

For example, the user may be enabled, when designing and setting up a clinical trial, to efficiently generate cartridges for each diagnostic lab involved in their trial. The cartridges will integrate and validate pharmacokinetic and pharmacodynamic data, collected from the multiple diagnostic labs during clinical trials, for internal analysis by the user's research and development team.

The cartridge generation system will enable diagnostic companies to streamline service to their customers. These companies will generate cartridges to service a particular customer's needs, and will use these cartridges for integration and validation of the pharmacokinetic and pharmacodynamic data generated by their multiple diagnostic testing labs for that customer.

Mechanism of a Translation Engine for Generating Translation Cartridges

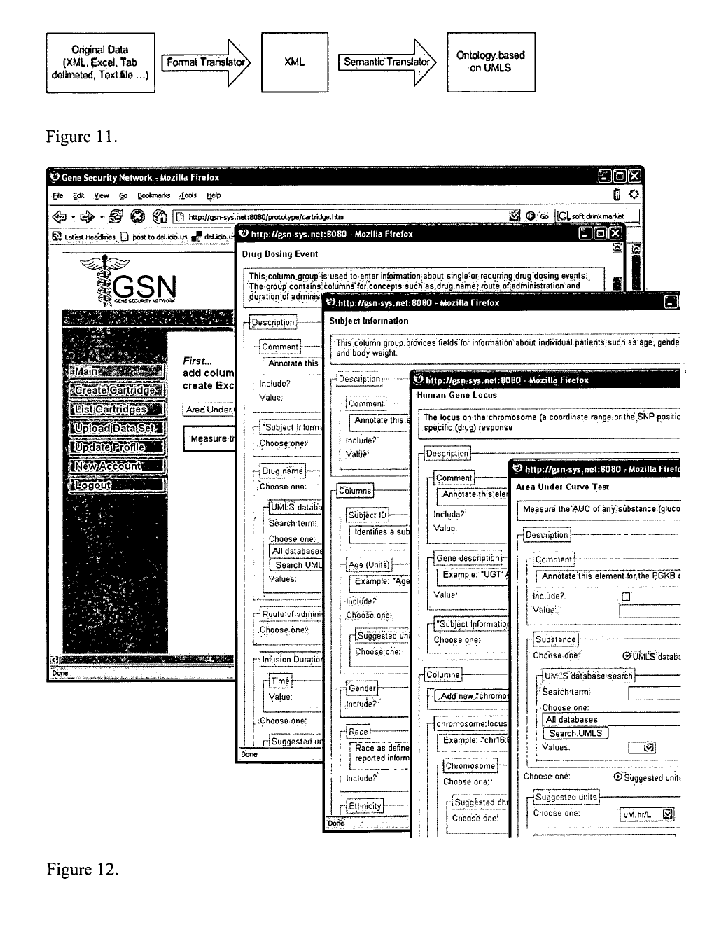

The data translation cartridge (see FIG. 11 for flowchart of translation process) is a computer based algorithm that can extract data from a set of electronic records with a wide variety of formats and fields, and translate those data into the appropriate location and format in a standardized ontology. The cartridge for a given data set is created using a cartridge generation program and with the help of input from a user who guides the program to make the correct links between the fields in the source dataset and the fields in the standardized ontology. The cartridge may have the following four components: a format translator, and semantic translator, a set of validation rules, and a set of predictors.

A format translator is a component that can take an input source and convert it into a standard computer language, such as XML. Input sources can be many formats, for example: database tables (SQL), HL7 documents (a common interchange format for EMRs), Excel spreadsheets, text based data (CSV, tab delimited), and other XML input. In one embodiment, the source data is converted into an XML document which is flattened into records and/or fields (for relational data like SQL, Excel, CSV). Note that the format translator does not interpret the data, but just reads it in and performs a non-semantic conversion to XML.

The semantic translator is responsible for converting the data itself into CSO concepts identified by System IDs. SYSTEM IDs are concept IDs fashioned after UMLS concepts and utilize the full UMLS concept hierarchy (e.g. a SYSTEM ID may be a synonym of a UMLS concept or can be a relation between two other UMLS concepts, or a mixture) The semantic translator reads the XML output of the format reader and converts each field of each record into the associated SYSTEM ID. It does this using a mapping from the original identifier to a Ssytem Identifier

In one embodiment of the invention, when a user needs to create a new cartridge, he selects the format reader and semantic translator that are appropriate for the given data set, and configures them both.

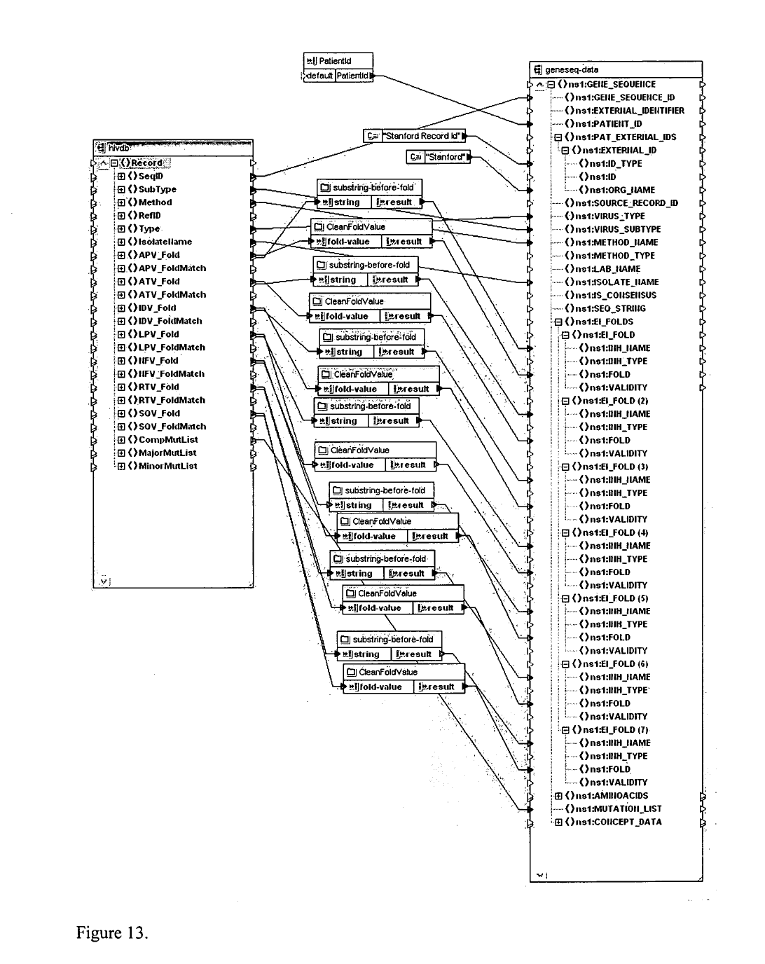

“Configuring” the dataset parser can be very time consuming, so two separate tools have been created to speed up the process. The first implementation of the semantic translator is a web interface for creating cartridges based on a CSO (see FIG. 12). When the user is not tied to legacy tables, or spreadsheets, then the easiest way to produce a semantic translator is by using a Context Specific Ontology (CSO). This lets the user create a new cartridge with guided contextual menus. The tool also produces spreadsheet templates based on the cartridge, and it includes embedded UMLS tie-ins. The second implementation of the semantic translator is an XSL Transform (XSLT) using Altova MapForce (See FIG. 13). In this implementation, users can create a mapping from local IDs to SYSTEM IDs. The mapping includes a small library of functions for data manipulation. There are also custom implementations of the semantic translator, and these can be implemented in Java.

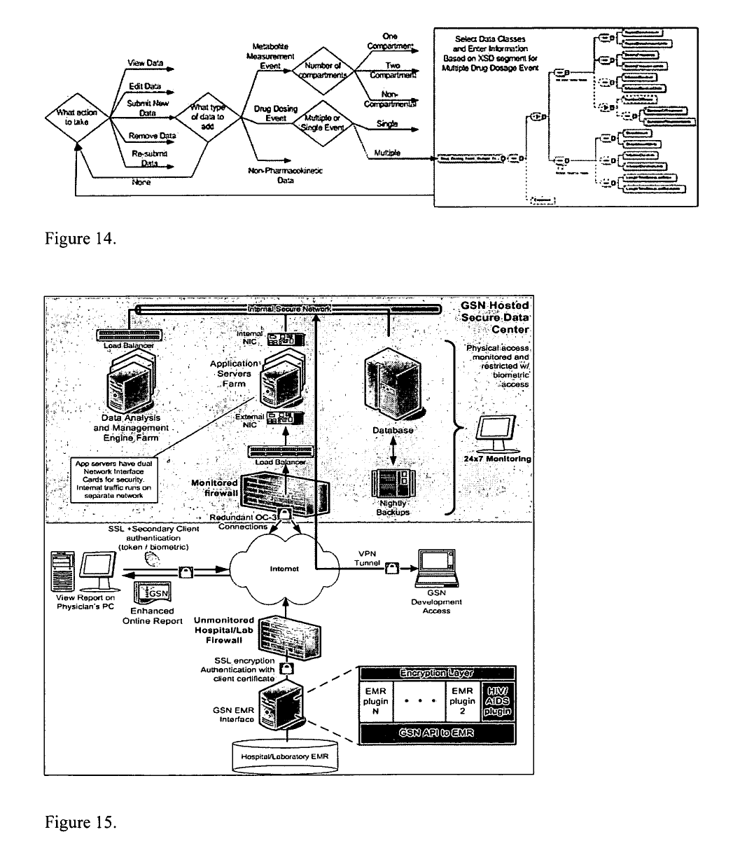

FIG. 14 illustrates a small subsection of the decision flow by which a researcher is guided to add data classes to accommodate local pharmacokinetic data. Up to the point that the researcher selects the element “Multiple Drug Dosing Events,” the figure only indicates a subset of high-level decisions by the researcher, but more information is entered—with more flexibility—than is shown. In the last steps, rather than show the decision flow, the figure illustrates the segment of the XSD schema for the element Multiple Drug Dosing Events, upon which the decision flow is based. Each series in the schema [symbol] involves a series of data class selections by the researcher, every choice in the schema [symbol] involves selecting elements from a pull-down menu, and every leaf element [symbol] involves either meta-data entry or selection from a pull-down menu. Attributes associated with each data class in the schema describe whether the data element is used to refine the headings of the Excel template, to define one of the columns in the template, or simply to guide the class selection process.

Data Integration

In one embodiment, after a cartridge has been created, the data is then integrated into the standardized database.

Data Protection

In one embodiment of the system, the software may contain an Encryption Layer that ensures that all data is transmitted with SSL encryption. The software also manages authentication with a client certificate to ensure that no third party can access the system. The aim is to ensure that the data submitted from an organization was not altered and its source can be confirmed. To achieve this, the system will use private and public keys. Navigating the encryption layer will consist of the following:
(a) When the data is submitted the system will create a hash (before encryption hash) of the full data file.
(b) The hash will be encrypted with the user's/submitter's private key.
(c) Once the data is received it will be decrypted using the user's/submitter's public key.
(d) The new hash will be created (after encryption hash) and compared the first hash (before encryption hash)
(e) If the hashes are identical then it can be confirmed that the data has not changed and the source of the data can be confirmed.
   The goal of these measures is to enable secure online reporting that the treating physician can access, which includes patient identification information so that the treating physician doesn't have to have separate lookup key for patient data, without in any way compromising privacy of the patient info.
   Part 11 Compliance

In one embodiment, the system may be compliant with the FDA's Electronic Record Rule (21 CFR PART 11), which regulates how pharmaceutical companies author, approve, store, sign, and distribute records electronically. When the system is updated with information, the system authority must know who updated the system, when it was updated, and what was changed. In addition, the system must be secure to prevent the possibility that an unauthorized party could have updated the record by hacking into the system.

Building an Interface between EMR and the System

To use the integrated and validated clinical trial and diagnostic test data to personalize therapy for a patient, without requiring the physician to manually extract and submit a large amount of additional data, it is necessary to automatically collect the relevant data from a patient's medical record. In one aspect of the invention, an electronic interface can be designed between the system and medical record systems, such as Cerner, a hospital-based electronic medical record system, to pull relevant patient information from the EMR for enhancement of diagnosis and treatment. To make a safe, useful product for hospital laboratories, the architecture of the system may deal with sensitive data under the rules and regulations of HIPAA and the FDA. The secure system architecture may also be part 11 compliant so that online reporting can replace paper records.

In one aspect of the invention, software that resides in a server may be deployed at a hospital, termed the Electronic Medical Record (EMR) Interface. The software may contain three layers: i) an Application Programming Interface (API) to the EMR in order to enable data extraction, ii) a disease specific EMR plug-in (such as for colon cancer) which uses the API to extract the data from the EMR that is relevant to the context of the disease, and iii) an Encryption Layer which ensures that all XML data is transmitted with SSL encryption and manages authentication with a client certificate to ensure that no third party can gain unauthorized entry into the system. Additional plug-ins may be designed for as many diseases, conditions or phenotypes as needed. The system will be designed for efficient implementation at new hospitals, using different EMRs (see FIG. 15).

The API enables data extraction. During format translation, the cartridge will extract the current and historic genetic sequence data, current and historic laboratory data (e.g. bilirubin levels), and the current and historic clinical status data available in the EHR System for incorporation into the standardized ontology. The cartridge and the ontology will also be extended to accommodate more fine-grained clinical status information as additional correlations between genotype and phenotype are derived.

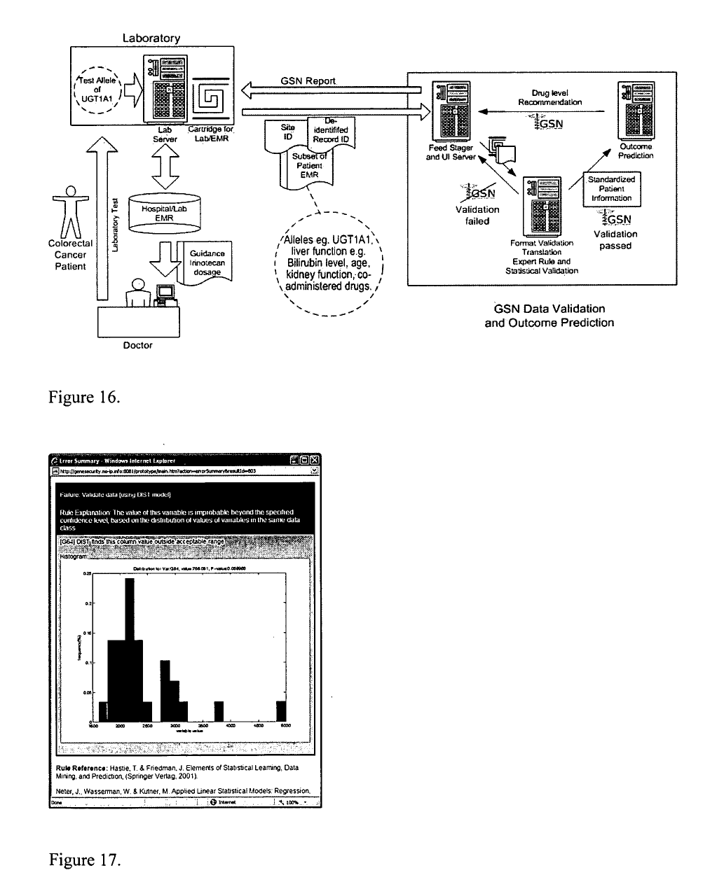

FIG. 16 illustrates the functionality of a cartridge implemented for a hospital laboratory. The operation of the cartridge will be similar to that described previously. It will include a format translation to convert data into XML and a semantic translation to convert the XML data into the format of the ontology standard. The data will be validated with format rules, expert rules, and statistical models as described. The key differences between the laboratory cartridge and the cartridges previously described is that the format translation for the laboratory cartridge will be implemented using a JAVA plug-in that accesses data in the EHR via an Application Programming Interface (API). A tractable subset of data that is relevant to the disease being addressing can be extracted.

Data Validation

The fidelity of the data that is integrated into the unified database is crucial for the accuracy of the resulting predictions, and thus the efficacy of the system. Given the disparate nature of potential data sources there are many sources of errors. Fortunately, the errors that are most likely to most affect the analyses of the data are those which fall significantly outside the expected bounds, and are therefore the errors that are easiest to detect. Consequently, it is important that all data uploaded into the standardized database undergo thorough validation to ensure that the phenotypic and clinical predictions are as accurate as possible.

In one embodiment, two types of relationships are layered onto the standardized ontology for automated data validation: i) expert rules associated with the standardized data classes, which check for errors, inconsistencies, or violations of established methods of data collection and clinical care, and ii) statistical relationships, which are parameter-based statistical models that relate the standardized data classes.

Expert rules are algorithms for checking the integrity of the data based on heuristics described by domain experts. Relationships are implemented as software functions that input elements of the patient data record and output a messages indicating success or failure in validation. Simple rules for the pharmacokinetics data include checking that all key data fields, such as the elements necessary to describe a metabolite measurement, are defined in the patient data record. More complex algorithms include assessing the possibility of laboratory cross-contamination of sequence data by checking correlation with previous samples. Expert rules may also encode best practice guidelines, such as those of the WHO, for collecting patient data and for clinical patient management. Examples include such considerations as ensuring drug dosing levels are within the acceptable range.

Statistical models describe relationships used to calculate the likelihood of data in a patient record given data about prior patients with similar characteristics. The statistical validation rules are essentially prediction models for which empirical confidence bounds have been computed using known techniques. New data that violates the confidence bounds is flagged as potentially erroneous. In their simplest form, statistical rules check the data values against the distribution of validated data that is described by the same segment of CSO-compliant XML that characterizes the meaning, format and context for the data. Data that is inconsistent with the distribution of existing data, beyond some specific confident limit (e.g. 95%) is flagged. Data can also be statistically validated for self-consistency within a record, using regression models that associate the computable data classes within a record. The techniques for generating these models are described elsewhere, either in this document, or other documents whose benefit is claimed above.

It is important to note that algorithms used for prediction can be used for validation of data as well: the concept of outcome prediction is essentially determining a most likely unknown outcome with a certain range of confidence based on a set of known outcomes, while validation is using a similar set of known outcomes to determine the a similar set of likely outcomes with a range of confidence, and determining if the piece of data under scrutiny lies within that range. It should be obvious to one skilled in the art how to adapt these algorithms for use in validation.

The researcher manages validation errors record by record by discarding the record entirely, editing the data for re-validation, or overriding the error. Once each record is validated, the data is pooled with likewise described data (from same and other cartridges) to automatically train phenotype predictors.

Once the data is contextualized in a computer-readable format, it is possible to compare data that is described by the same segment of XML (i.e. one that has the same system ID). Most simply, for data validation, it is possible to generate a distribution of data for a particular data class (system ID). More advanced regression models can be used that check self-consistency of a record, such as linking HIV/AIDS genetic sequence with resistance to reverse transcriptase inhibiting drugs.

Each data validation or prediction function is associated with a particular system ID to be predicted, and with a cartridge to input a set of IVs (each associated with a system ID) to be used for the prediction. The models for data validation will be automatically generated as described above. However, the models for data prediction (this function is not central to the integrity of the system and is optional) will always include human expert intervention to validate the model. Expert intervention will also be necessary to describe thresholds for the system IDs to be predicted and the actions to recommend for each range between thresholds.

EMR Data Considerations for Validation

The validation rules can be applied to data that originates from many sources, including a spreadsheet, or a patient's electronic medical record. To blindly validate all EMR data for statistical validity is not meaningful. In one embodiment of the invention, as each cartridge is built, a translation table can be included from CSO leaf nodes to EMR elements. After uploading only the relevant measurement information from the record, validation can proceed as previously described. Certain architectural elements can be added to support EMR data. FIG. 11 shows the stages of translation (format, and semantic). One of these elements may be new JAVA format translators to accommodate one of HL7 or direct ODBC connectivity; another may be a new semantic translator which includes a mapping from CSO leaf nodes to EMR identifiers.

Statistical Rules

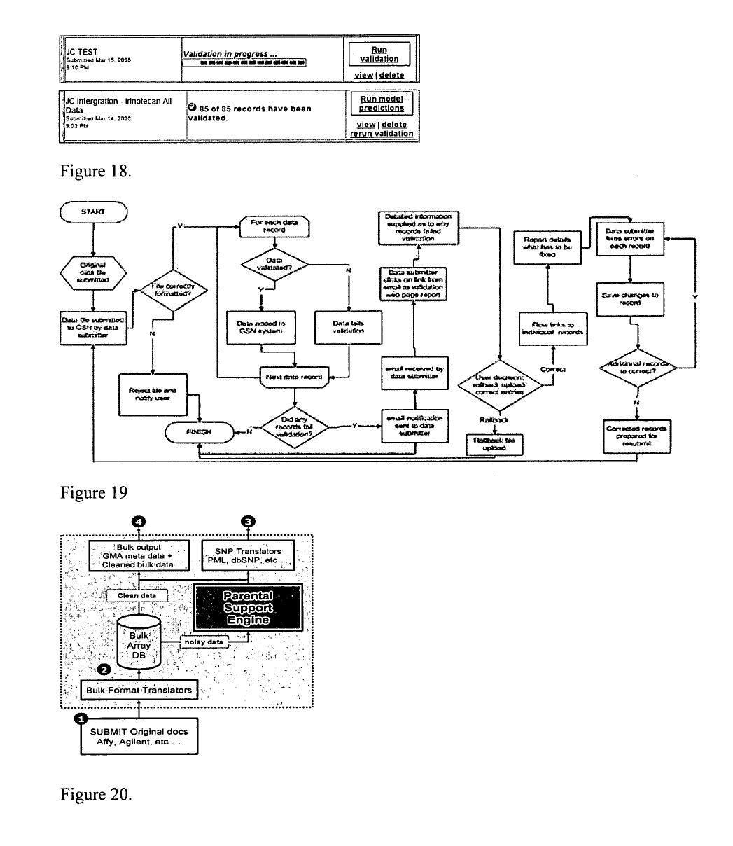

In one embodiment of the invention, when a particular data set is selected or newly submitted for validation (FIG. 17, top), the system site may show the results of the submission (FIG. 17, bottom) and let the user review all failures and warnings for each record. Statistical methods may be used that check the distribution of the variables within a particular column or data class and do not use any regression models to link variables statistically. These methods are used for both categorical variables and numerical variables. In both cases, variables that lie below a particular user configured probability level (e.g. 5%) are flagged. When a particular error is selected, the system shows an error details page which explains the error. In the case of numerical variables, a histogram is shown (FIG. 18), with the specified confidence bounds in black and the outlier in grey. In the case of categorical variables, a bar chart is shown with the bar corresponding to the offending variable in grey. For numerical values, the confidence bounds are empirical bounds based on the histogram and are not based on fitting the data to a Gaussian distribution.

The distribution against which variables are checked is based on the system ID associated with that variable and an XML description stored in the database. A single directory contains a set of mat files, each of which is associated with a particular system ID. These files are loaded and augmented with new counts each time data associated with a particular system ID is submitted and validated against existing data. If any changes occur in the meta-data describing a variable, a new distribution is created for that variable. If the cartridge is new, data are checked against other data in the newly submitted file. If the system ID is new, mat model files are created. The distribution is created with the new data, the data outside the 95% (or whatever confidence bound) is flagged, and the distribution is created again with all flagged data removed.

The user can change or corroborate flagged data. The system gives the user the opportunity to clean the data for purposes of sharing it. Once data passes validation, the user can see the data translated from his organization's particular format into a global UMLS-based format.

In one embodiment, a record is kept of the entity responsible for validating the various pieces of data. As the validation of data that is initially flagged is a human-based process, there is room for error. By keeping track of the entity responsible for validating various pieces of data, if it is discovered later that a certain validator had an unacceptable record of validation, those pieces of data could be revalidated by a more reliable individual. In addition, if significant decisions are to be made based on analysis of a given set of validated data, it may be of interest to the decision makers who was responsible for validating the relevant data.

In another embodiment, data validation checks are continually re-run as more data is integrated into the system. Since some validation rules may be based on expected statistical distributions, and those expected distributions are based on the data present, as more data is integrated, those expected distributions may shift. As such, pieces of data that had previously been validated may become subject to question. An automatic validation check could flag the data that has become questionable for further scrutiny.

The Decision Flow for Data (Re)Submission and Validation

In one embodiment, the data validation process is illustrated by the flow diagram in FIG. 19. When data is submitted, it is held in a staging area, where it is validated against all relevant rules. If all rules validate correctly, the data is added to the patient database. If a rule fails, the new data is flagged, and the text message associated with the failed rule is added to a list of reasons for the failure. If any rules from a given upload batch fail validation, the entire batch is held in quarantine.

Whether or not data fails validation, the submitter receives an acknowledgement of the data upload, how many records were uploaded, and whether any records failed validation. If records fail validation or generate warnings, a hyperlink is included to direct the user to each record that requires correction. Each record that failed validation links to an error details page displaying details of the record and a list of warnings or error messages. On this page, the user is able to update the record, remove the record from the set, or override the error message. When the user has finished updating the invalidated records, he/she can resubmit the entire file.

Statistical Methods to Predict or Validate Phenotype/Outcome from Limited Data:

Applying Ockham's Razor to Model Underdetermined or Ill-Posed Data

A main purpose of aggregating data into a standardized ontology is to allow for better, more accurate medical predictions to be made that will enhance the lives of people. Some techniques and methods which may be used in this context are described in detail in patent application Ser. No. 11/496,982, filed Jul. 31, 2006, whose benefit is claimed herein. Note that these methods which were previously described in the context of predicting phenotypic and clinical outcomes may also be used for the purpose of data validation.

Sparse parameter models are generated for underdetermined or ill-conditioned genotypic-phenotypic data sets. The selection of a sparse parameter sets exerts a principle similar to Occam's Razor: when many possible theories can explain the observed data, the most simple is most likely to be correct. In one embodiment, support vector machines may be used to create non-linear models, or LASSO techniques may be used to create linear models, both of which are trained using convex optimization techniques to make the models sparse. In another embodiment, models may be based on contingency tables for genetic data that can be constructed from data available in genomic databases. One focus of the patent whose benefit is claimed above is the modeling the response of HIV/AIDS to Anti-Retroviral Therapy (ART) for which much modeling work is available for comparison, and for which data is available involving many potential genetic predictors. These techniques are able to predict viral response to anti-retroviral therapy more accurately than previously published methods.

Implementing the Statistical Rules for Prediction

In one embodiment of the invention, generic functions may input a text file containing a systemID to be predicted together with a list of systemIDs to be used for the prediction. Also included may be thresholds for the systemID to be predicted, and the actions to recommend for each range between thresholds. The system goes through all permutations of models with the available data, cross-validating each, until it comes up with the best subset of predictors out of those chosen. If the solution is underdetermined solution, then number of variables must be more limited. For positive variables, log of the variables are checked as well. Having selected the best model, the result is generated with the prediction on a histogram against outcome training data, and an estimate of the CDF after the predicted outcome (i.e. bigger than x % and less than 1-x %).

Use of the Schema for Genetic Data

Genetic information represents a major class of data that will become increasingly important for clinical prediction as more genotypic-phenotypic correlations are discovered. FIG. 20 shows how, in one embodiment, it is possible to both internally translate and store bulk data from raw genotype measurement files, and provide external interfaces to retrieve data in well understood formats. The flow of the system is as follows: 1) The user submits original bulk documents from high-throughput genotyping systems (from Affymetrix, Agilent, etc . . . )—in the IVF context, for both the parents and embryonic DNA. The system will also demand from the user certain meta-data about the individuals necessary to describe the data and drive the system flow; 2) the genotyping data is translated into an internal binary format, suitable for large amounts of bulk data, and stored along with the meta-data from stage one. 3,4) When the user requests either a particular SNP value, or a copy of processed bulk data for storage, the Parental Support engine is invoked and data is cleaned.

There are a number of existing de-facto and emerging standards suitable for describing a single, or a small number of SNPs. No such format exists for bulk data. Attempting to use standards like dbSNP or PML for bulk data would be unwieldy. It is desirable to extend existing standards to support bulk array data that are practical, long lasting, and industry accepted, and to maintain the ability to readily incorporate other standards that become available. Note that PharmGKB is currently engaged in a substantial effort to represent high-throughput genotyping data in the public domain. It should be obvious to one skilled in the art how other types of data can be integrated into a standardized ontology using these methods.

Implementation of the System to Generate Enhanced Diagnostic Reports

In one embodiment, the system may be designed to use the integrated data to make predictions regarding a particular individual, and then to generate an enhanced report regarding the individual. In one embodiment of the invention, the data is analyzed to give phenotypic predictions, and those predictions organized into a report for the purpose of effectively disseminating the relevant predictive information to the people who can best use it, i.e. physicians, clinicians, and researchers.

The report may contain predictions and/or likelihoods of various phenotypic, clinical or medical outcomes given various actions. For example, in the case where a patient has colon cancer, a physician may be interested to know the likelihood of cancer response to a given pharmaceutical product and treatment schedule given the phenotypic and clinical data of patient, and/or the genotypic data of the patient and/or the cancer itself. In this case, the system described herein may make these predictions, and generate an report containing the most germane predictions for the attending physician in a way that it is most likely to benefit the patient.

In one example, the system may generate a complete diagnostic report in order to aid doctors in selecting the optimal therapy patients suffering from an illness or condition. This report may have the following features:

(a) It may apply algorithms, possibly those described in a cross-referenced patent application, to produce a prediction. The prediction may be generated with the best available model for the subset of IVs available for that patient.

(b) It may include graphics of genetic mutations and laboratory measurements found to be relevant to predicting drug response and an indication of the strength of their contribution to the model.

(c) It may include confidence bounds for the prediction of key pharmacokinetic and clinical outcomes based on the models.

(d) Whenever diagnostic assay tests are available and validated, the report may include this data.

The physician or other agent may be able to view the enhanced report online by means of a web browser. S/he may need to log on to the system with a username and password. For enhanced security, the physician may also be required to enter a code from a hardware token located at their computer upon logon.

Each deployment of an enhanced reporting system for a new customer may involve:

(a) Provisioning the application for enhanced reporting in the system authority's data center

(b) Provisioning the EMR Plug-in in the EMR Interface to extract the relevant information from the EMR.

(c) Setting up an account for the client hospital to enable access to online reports.

Automatic Generation of Enhanced Reports

In one embodiment, the system can be configured to automatically generate enhanced reports for certain patients at regular intervals, or when new, pertinent medical information is integrated into the system. Medical science is a field where rapid advances are the norm, and where large volumes of data are constantly being generated. Consequently, it is possible and even likely that a given set of predictions may change as the knowledge in the field and/or the data in the system changes. As physicians and clinicians are not able to keep abreast of all changes, it may be beneficial for enhanced reports to be generated regularly and disseminated where appropriate to keep patient care up to date.

The Database Architecture and Interface to the Application Server

To make the code base robust with regards to database evolution, in one embodiment the middleware interfaces to the database by means of an API. This API is accessed by the DAME, the feed validator, the feed parser, and the user interface server, which are currently implemented as separate modules in a single application server. All data validation rules and prediction models are implemented using an object model where each rule is encoded inside a separate code class in JAVA. For statistical models, JAVA calls compiled MATLAB executables created with the MATLAB COMPILER.

Hardware and Software Details

In one embodiment of the system a 32-bit Linux server system is deployed on two 32-bit computers powered by Intel x86 CPUs. Network equipment includes routers, switches, and load balancers from Cisco Systems. The database and data warehousing tools are from MySQL (v5.0). The web server runs Apache and uses Tomcat version 5 as a servlet container. All middleware logic is built using a Java 5.0 framework using Spring Framework (version 1.2) as a lightweight web framework, and Hibernate (version 3.1) as an object/relational persistence platform. The DAME server is implemented using Matlab. The Matlab service is made available for internal use and testing through a secure web service with its own well-defined, internally developed APIs.

In one embodiment of the system a tool will guarantee the security of access to data at many levels. Password access is required to view and edit data, and if necessary, user-level voluntary and involuntary password sharing will be addressed by biometric authentication such as iris scans. System-level vulnerabilities are protected with a multi-layer security architecture. All HTTP traffic from internet clients is encrypted using 128 bits SSL encryption. Furthermore, all datacenter traffic is limited to developers, administrators and other groups approved by a centralized authority, and is secured though encrypted SSH tunnels over non-standard port. The firewall blocks requests on all ports except those directly necessary to the system's function. Each application server has two network interface cards (NICs) and exists simultaneously on two sub-nets, one accessible from outside the firewall and one not. The application server may be blocked from the application server by another firewall and also exists on two sub-nets, one for communication with the application server and one for communication with the database. An intruder would have to break through the firewall and gain access to two layers of servers before attempting an attack on the database. Access to each server is logged, and repetitive unsuccessful logins and unusual activities will be reported as possible security attacks.

The system datacenter is protected with FireSlayer, an anti-Denial of Service (DOS) technology. This feature automatically allows the maximum legitimate traffic while rejecting illegitimate traffic. To further protect the server, it may be useful to use an intrusion prevention system, such as TippingPoint, that continuously filters any malicious packets to protect the server from vulnerability and exploit attacks. The servers are also periodically scanned with Vulnerability Scanner, which will scan the entire server to ensure that it is up to date with the latest patches.

In one embodiment an existing un-monitored firewall at the hospital/laboratory facility can limit access to the EMR Interface; a monitored firewall at the system authority's data center can limit access to the Application Servers. The Application Servers, Data Analysis and Management Engine (DAME), and Database may all reside at a hosted facility. This can provide 24×7 system monitoring, nightly backups, and load balancing for the Application Servers and DAME. The system may use single Linux-based PCs for the Application Server and DAME. The Application Server may exist on an external and an internal Network Interface Card (NIC). The internal network will be accessible by developers from the outside by means of a VPN.

Encryption—Digital Signature

In one embodiment, data that is submitted may have security features built in. The aim is to able to claim with certainly that the data submitted from an organization was not altered and its source can be confirmed. To achieve this, the system may use private and public keys. When the data is submitted the system will create a hash (before encryption hash) of the full data file. The hash will be encrypted with the users/submitters private key. Once the data is received it will be decrypted using the users/submitters public key. The new hash will be created (after encryption hash) and compared the first hash (before encryption hash). If the hashes are identical then it can be confirmed that the data has not changed and the source of the data can be confirmed.

Other Contexts

The system described in this document could equally effectively used in a cariety of contexts. For example the standardization, aggregation and validation of data could be done in the context of drug discovery. The data could originate from a research project focusing on targeted drug discovery by a pharmaceutical company. In this context the data fields may include a series of related molecular structures, and the related impurity data, in vivo and in vitro assay data, details of the in vitro assay protocol, details of the animal model used in the in vivo assay, toxicology studies, formulation research, and/or pharmacokinetics data. The analysis of the data may be able to uncover important relationships between molecular structure and important pharmacological properties such as structure-activity relationships, metabolic-toxicological trends within a class of compounds, or absorption-bioavailability trends, for example.

It will be recognized by a person of ordinary skill in the art, given the benefit of this disclosure, aspects and embodiments that may implement one or more of the systems, methods, and features, disclosed herein.

Example of Reduction to Practice

Example of an Implementation of the System

One embodiment of the system was alpha-tested by data curators of PharmGKB to integrate colon cancer data from PharmGKB. There are two key applications of the production system: i) streamlining the integration/validation of patient data from clinical studies and ii) making outcome predictions based on the integrated data. For each potential application, the functionality of the system was demonstrated by researchers, clinicians, and bioinformatics experts, who were asked to complete a detailed survey. Several rounds of testing was completed, with modifications being made throughout the process.

Step-By-Step Example of Model System

What follows is an example of the steps that may be necessary for a user to create a data translation cartridge in one embodiment of the invention. It is important to note that there are many ways that this invention may be implemented, and this example is only meant to demonstrate one possible working configuration of the system. It is important to note that this is not meant to be an exhaustive example of all the possible web pages, interfaces, dialog boxes, spreadsheets, or other elements of the system. In addition, any one of these steps can be used separately, in combination with other steps, or in combination with other steps of other embodiments of this system, or with other systems.

Step 1: Creation of a New Cartridge

This step details how a user would create a new cartridge. Users must have data to integrate into the system. The user will utilize a web interface to select elements from drop-down lists to build a data translation cartridge that contains one column for each element. Each element should map to a data element the researcher wants to upload.

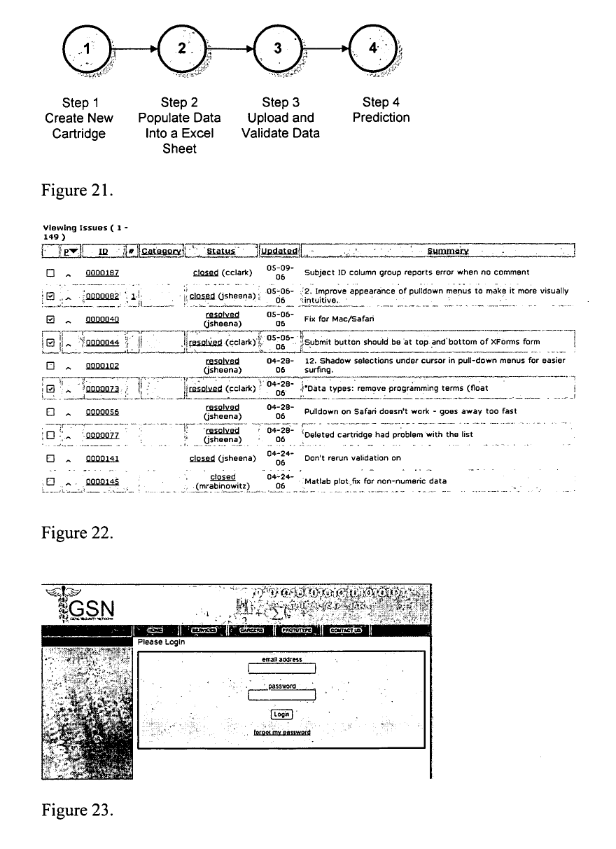

The components of the system (FIG. 21) include creation of a new cartridge, creation of a local Excel spreadsheet for data entry, upload and validation of the data entered into the spreadsheet, and can also include prediction of clinical outcome based on statistical models using all previously integrated data. Each functional component was tested. Mantis Bug Tracking System was used to systematically record, prioritize and address internal and external user comments and to correct system errors (FIG. 22).

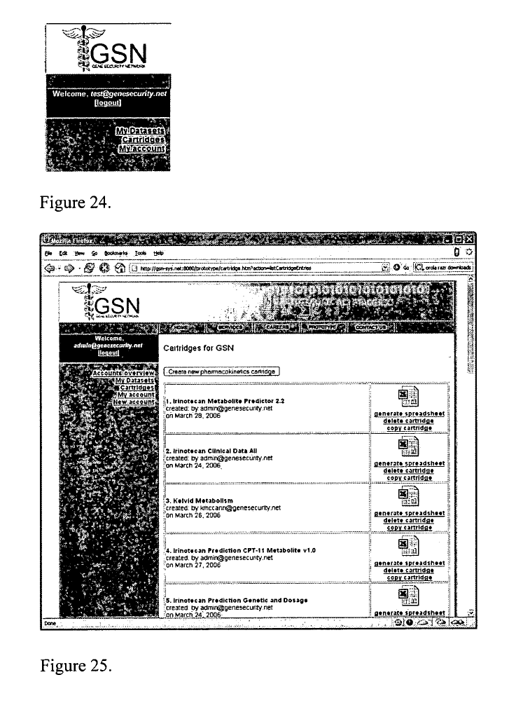

In one implementation of the system, a working cartridge generation engine has been designed. The process of using the system is shown in detail here. First, the user will go to the appropriate webpage hosted by the system authority, type in a username, and a password. The login page is shown in FIG. 23. At the login page, all users must login with an email address and password. After login, and once authenticated, the user will see the welcome screen, shown in FIG. 24, which displays a menu for viewing summary status of all data sets from the organization that have been validated in the past and all of the cartridges that have been created to integrate that data into the system.

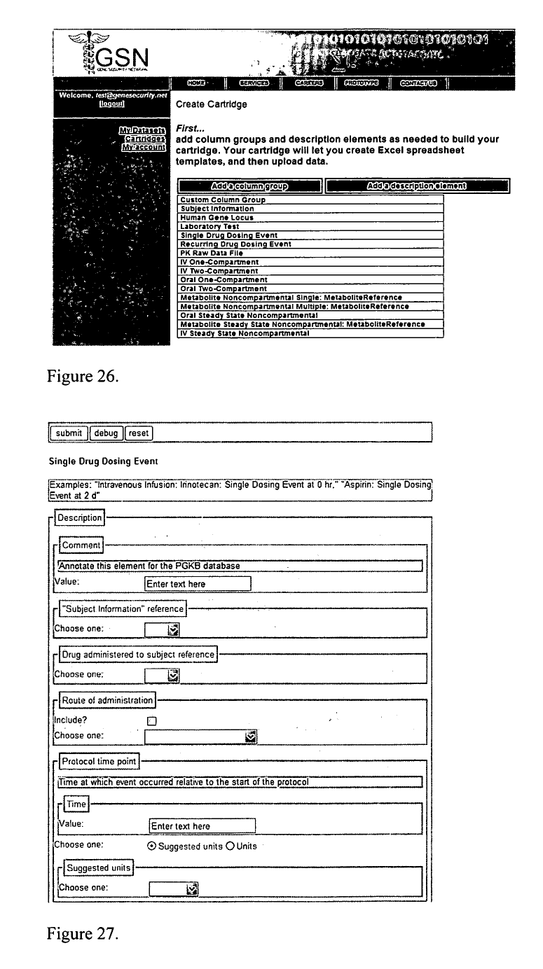

The use may first select “Cartridges” to get to the cartridges page, shown in FIG. 25. The user may then click on the “Create new Pharmacokinetics cartridge” button to get to a cartridge creation page shown in FIG. 26. A web interface guides users through cartridge creation. The web interface is implemented by JAVA code that processes any properly formatted XSD schema and automatically generates a series of pull-down menus and fields for entering information. Consequently, the XSD completely dictates how the researcher is taken through a series of class selections and information entries.

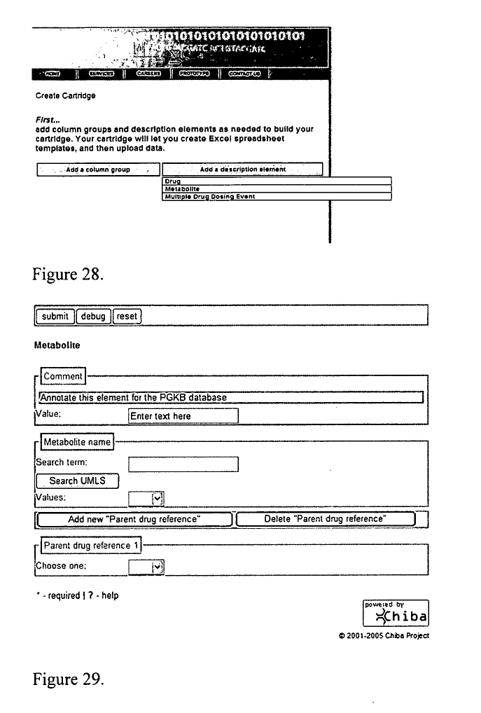

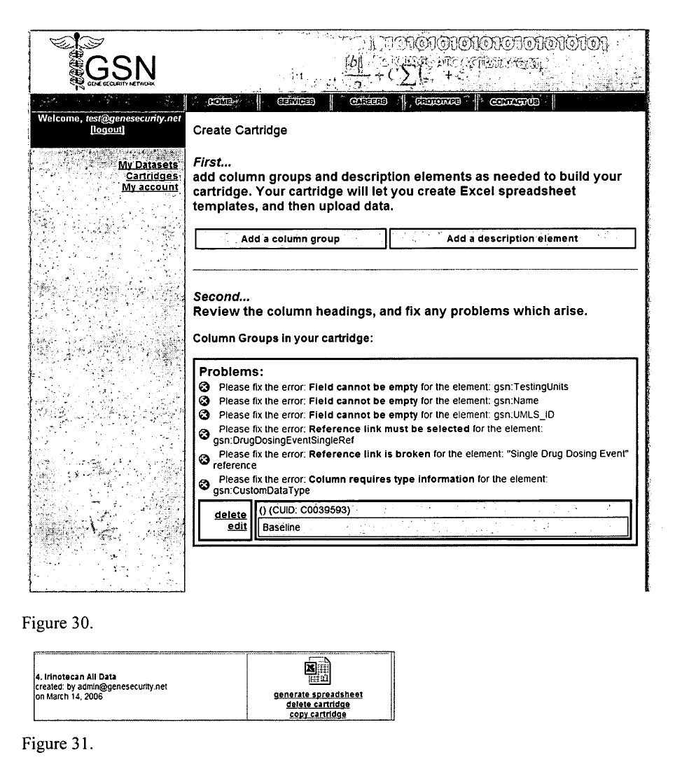

The user may choose the relevant data classes to accommodate his or her local data. In order to add a particular data class, such as “Subject Information” or “Single Drug Dosing Event”, the user clicks on the “Add a column group” and a drop-down menu will appear on the screen as long as the user holds down the “Add a column group.” Once a column has been selected, the window shown in FIG. 27 will immediately appear for further specification of the data class. For example, “Subject Information” can include gender, race, and ethnicity, among other qualifiers, but if the user only has gender information for his or her patients, s/he can choose to include gender and exclude race and ethnicity. The user may then click on the “Add a description element” as shown in FIG. 28. Once a description element has been selected, the window shown in FIG. 29 will open. After entering the required information, the user may click the “submit” button, and move to the next step. The system will require the user to correct selection errors, as shown in FIG. 30. This can be done by clicking on the “Edit” button. The system will check that the elements selected pass certain rules. The rules ensure that the cartridge created is of an acceptable format and contains useful data. Warnings are generated if the elements selected do not meet the rules. The user must correct the mistakes to remove the warnings. The system will inform the user when a valid cartridge is created. Once the cartridge is correctly built, the process is complete. Enter a name for the cartridge and click on the “Save” button.

The web interface used to select/specify data classes is implemented using Chiba server-side Xforms. XSLT is used to translate the CSO into an XForms documents implemented as X-HTML. Java code is used to expand all enumerations in the CSO into a list by querying the UMLS metathesaurus database. The lists are stored in separate files and are hyper-linked into the XForms document. The XForms, in creating the web interface, pull the enumerations from the file created by the JAVA code.

Once the user has selected data classes, XForms generates an XML document that contains all of the user class selections. This has a set of redundant information related to XForms, which are cleaned by XSLT to make an XML containing all the specified class information. This XML is then be acted on by an XSLT to generate the Excel spreadsheet template in the form of an SML document. In addition, the cleaned XML is acted on by XSLT to generate the Cartridge XSD. This contains all of the class associations and other information to validate and parse the information that is submitted according to the Excel spreadsheet template.

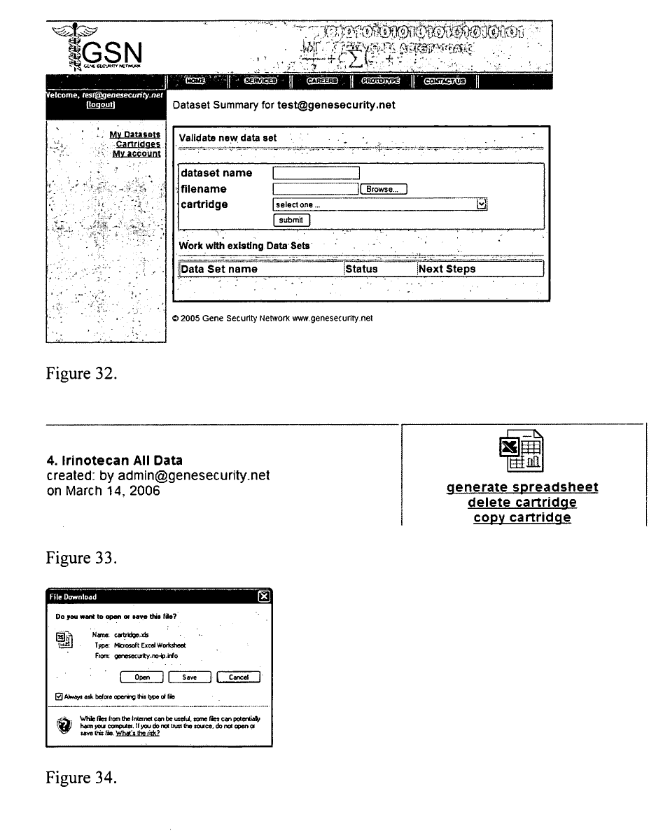

Once the user has created a cartridge, she is given the option to copy the cartridge for editing purposes (preserving the original cartridge), to delete it entirely, or to download an Excel spreadsheet for data entry (FIG. 31). The user cuts and pastes data into the Excel template and saves the data locally. For data submission to the central database, the user creates a name for the data set to be referenced thereafter in the central system, selects the local Excel data file, chooses the relevant cartridge and clicks “Submit” (FIG. 32). Appropriate plug-ins are loaded to convert the Excel template into the Data XML document. JAVA code inputs the Data XML together with the Cartridge XSD. The first step is for the Data XML format to be validated using the Cartridge XSD. The JAVA code will then use plug-ins to convert certain incoming data formats to outgoing data formats. Once all data has been converted into the correct format, the data is stored in the database in CUI-value pairs that are also associated with the ID for the Cartridge XSD, which is saved in the database as a document. The Cartridge XSD is written to a table in the database, in which all the relevant CUI's for the cartridge are stored so that the full set of data from the Data XML can be pulled from the database by a SQL query.

Step 2: Populate Data into Excel Sheet

This step describes how a user could enter data into a spreadsheet and upload it into the system. It is assumed that the step 1 (above) has already been completed.

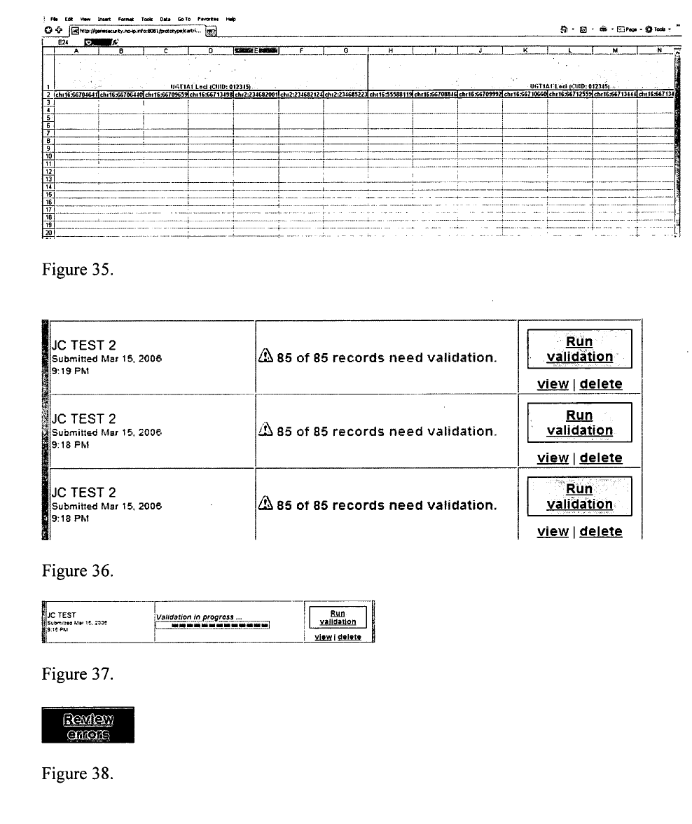

Back at the welcome screen, (FIG. 24) the user may select “Cartridges”, and on the cartridges page (FIG. 25), the user may select the cartridge of interest, as displayed in FIG. 33. By clicking on “Generate Cartridge” icon, the window shown in FIG. 34 will open and the user may select “save”. The system will open Excel and build an Excel spreadsheet with columns based on the cartridge. The spreadsheet will contain one column per data element, as shown in FIG. 35. The user may then paste data into the relevant columns in the spreadsheet. The Excel spreadsheet can be saved with a unique user-defined filename on the network or local hard drive.

Step 3. Upload and Validate Data

This step details how a user would upload and validate a data file. It is assumed that steps 1 and 2 have been completed.

Back at the welcome screen (FIG. 24) the user may select “My Datasets” to open the window shown in FIG. 32. The user may then enter name for the data file, click on the “Browse” button and select the file defined in Step 2 from the directory, select cartridge name defined in Step 1, and click on the “Submit” button. This will retrieve the Excel data file and upload the data to the system. The system will associate each element with XML metadata describing the context for that data. Basic data scrubbing is performed at this point, including checks that the column names are correct and that the data meets certain basic formatting requirements. The data file can now be found on in the “My Datasets” page. The status column (FIG. 36) shows the number of records and how many of them require validation.

The system can run validation on each record in the data set when the validation button is pressed. After clicking the “Run validation” button on the right side of the screen, a window such as the one shown in FIG. 37 will appear. Once the validation process has begun, the system performs a number of detailed steps to ensure that the data is not outside the expected statistical boundaries. If data is outside expected probabilistic bounds, it is flagged with an error or warning message, such as the one shown in FIG. 38. Once validation is complete, the results should be reviewed, and errors and warnings resolved. To do so, the user may click on the “View errors” button.

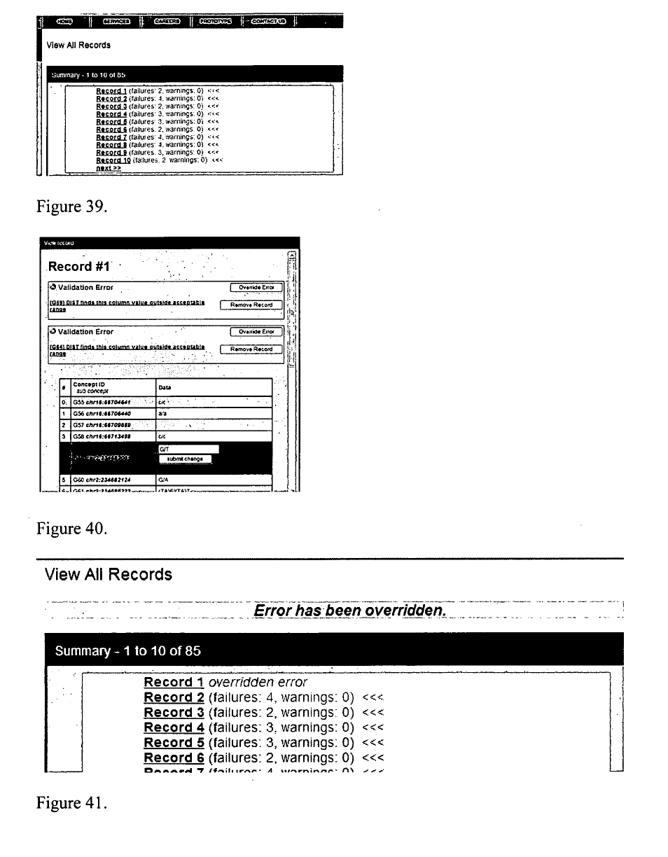

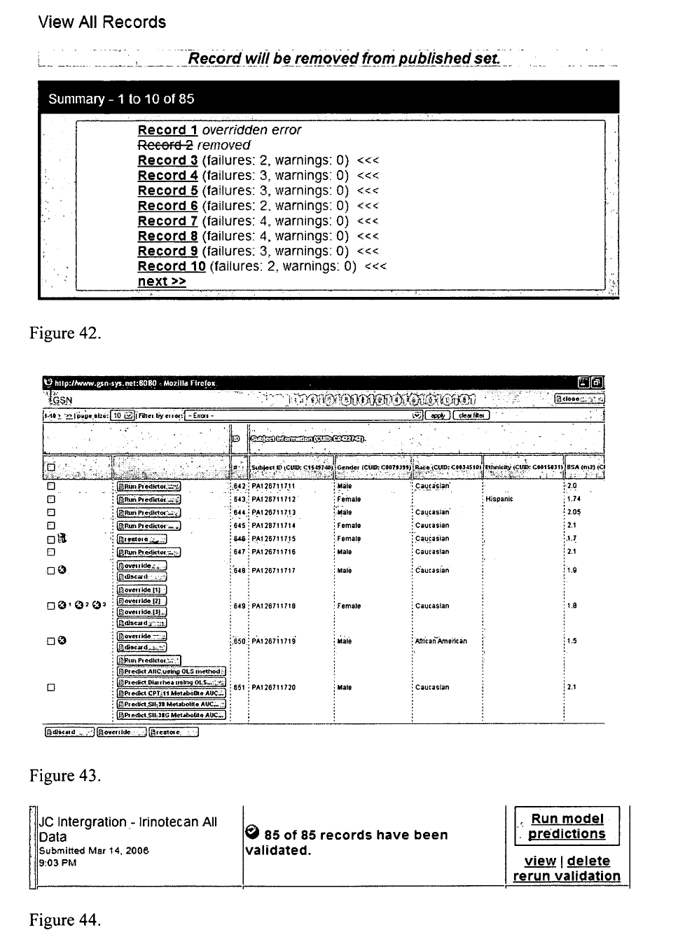

This will open a window and each record within the data file will be displayed (see FIG. 39). An error and warning count will be displayed for each record. Clicking on an record of interest will show the window in FIG. 40. These errors can be corrected or overridden as described here: To do so, the user may click on each record to (i) override the flag/warning message, (ii) remove the record from data set, and/or (iii) view the histogram illustrating data that is outside the acceptable range. The override option produces the message shown in FIG. 41. The “Remove Record” option produces the message shown in FIG. 42. The distribution view shown in FIG. 18 finds column values that are outside acceptable range. Once each record's errors and warnings have been resolved, the user may return to the “My Data” page. The number of records that require validation should have changed, and the user can view the list of validated record within the dataset (FIG. 43).

In order for the changes to take effect the user must click on the “Run Validation” button again and wait for the results. The results of this validation should produce fewer errors and warning messages. The user may continue in a loop of fixing errors and warnings until the data file is ready for final validation. If there are no longer any validation errors, when the user clicks “Run validation” button, a window such as the one shown in FIG. 44 should appear. All records in the data file should be validated.

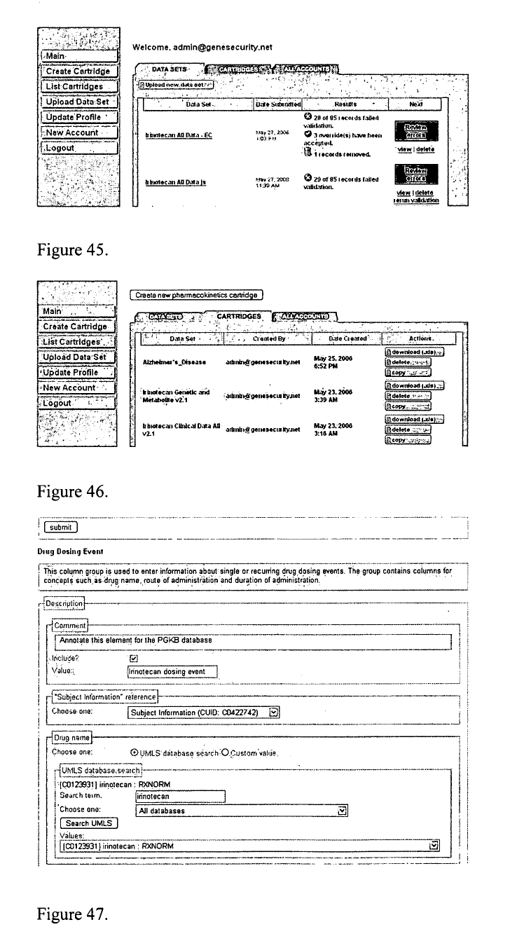

Some features that may be included in the system include an expansion of the user menu to include explicit tasks for users, such as “Upload Data Set”, and the implementation of a system of easily-readable charts and tabbed files such that an institution using the system can track use by its members and utilize the data sets most efficiently (FIGS. 45 and 46). After data submission and validation, the user may simultaneously view all of the records of a particular data set, sort the records by validation errors and correct all similar errors simultaneously if appropriate, run one of a number of outcome predictions (e.g. metabolite levels, diarrhea risk or neutrophil count) which were trained by the system, easily view details of validation failures, and discard or restore individual records or the entire data set (FIG. 47).

Step 4: Generate Prediction and Enhanced Report

In this step of this example, the focus of the system is to improve the treatment for colon cancer patients. The cartridge format translation is be implemented by a JAVA plug-in that accesses information from the EHR by means of Structured Query Language (SQL) queries. EpicCare, an EHR from Epic Systems Corporation, can provide an interface to the clinical data stored within the EHR, including laboratory data, via an application called Clarity. The Clarity system can then extract data from the production server and store it in a relational database on a separate, dedicated reporting server: the analytical database server. Storage in the analytical database server will enable the system engineers to implement the necessary SQL queries to extract the subset of information described above. EpicCare supports connectivity to the controlled vocabulary SNOMED (Systematized Nomenclature of Medicine Clinical Terms), which is one of many source vocabularies in the UMLS Metathesaurus. SNOMED's concepts, hierarchical contexts, and inter-term relationships are preserved in the UMLS Metathesaurus. EpicCare is used by over 140 healthcare organizations and stores the healthcare information of over 55,000,000 patients across the US.

An EMR colon-cancer-specific plug-in can use the API to extract the data from the EMR that is relevant to the context of colon cancer, including general subject information such as age, race and gender, and clinical or laboratory data such as kidney function and liver function assays (such as bilirubin levels), co-administered drugs, and SNP analysis of the UGT1A1 gene. The UGT1A1 gene encodes the enzyme UDP-glucuronosyltransferase, which is involved in breaking down Irinotecan. Specific variations in UGT1A1 can cause irinotecan toxicity. Variations in the UGT1A1 gene can be measured by the Invader UGT1A1 assay manufactured by Third Wave Technologies and marketed by Genzyme.

If possible, the data may be extracted along with the associated date stamp. The plug-in extracts the available data and converts that to XML. The data is then associated with a site ID, a record ID and a cartridge ID, encoded, and conveyed to the Feed Stager and UI Server modules in the Application Server. The associated cartridge is then used to validate the data format, to semantically translate the data into a format consistent with the Context-Specific Ontology (CSO), and validate the data with expert rules and statistical models. Any data that fails validation generates an online report that goes back to the lab in order for the data to be upgraded or corroborated, after which the data will be validated. The validated data is then rendered in standardized computable format based on the CSO.

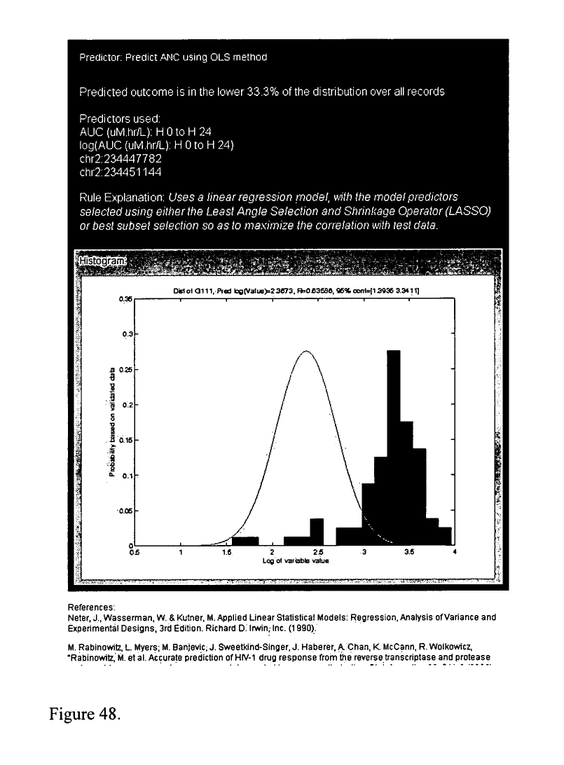

At this point it is possible to apply algorithms described elsewhere in this document, in cross-referenced applications, or from public sources to produce the diagnostic reports, and phenotypic or clinical predictions. The system may make predictions using outcome prediction models trained on data integrated from a plurality of sources, such as from PharmGKB, ongoing treatment records, or hospital-based EMRs. This system can input a patient's data gathered electronically from the EMR and relevant diagnostic tests. Enhanced reports may be generated for patients, in this case, those suffering from colon cancer, which will indicate to a treating physician the likelihood of various responses to various treatments or courses of action. In the case of colon cancer patients, the report may indicate whether treatment with Irinotecan is suitable for each individual. The report will include predictions and confidence bounds for key outcomes for that patient using models trained on integrated data (See FIG. 48). In the case of the colon cancer patients, the data may include clinical trial data, and/or patient genotypic, phenotypic and medical data. A physician may be able to view the enhanced report online by means of a web browser after logging onto the system with a username and password, and entering a secure code from a local hardware token.

Described here are some additional details concerning the inputs and outputs of the example enhanced report for colon cancer. Considerations are presented here (e.g. contraindications for treatment, dosing schedules, side effect profiles) for the production of a clinically useful enhanced report. Myelosuppression and late-onset diarrhea are two common, dose-limiting side effects of irinotecan treatment which require urgent medical care. Severe neutropenia and severe diarrhea affect 28% and 31% of patients, respectively. Certain UGT1A1 alleles, liver function tests, past medical history of Gilbert's Syndrome, and identification of patient medications that induce cytochrome p450, such as anti-convulsants and some anti-emetics, are indicators warranting irinotecan dosage adjustment.

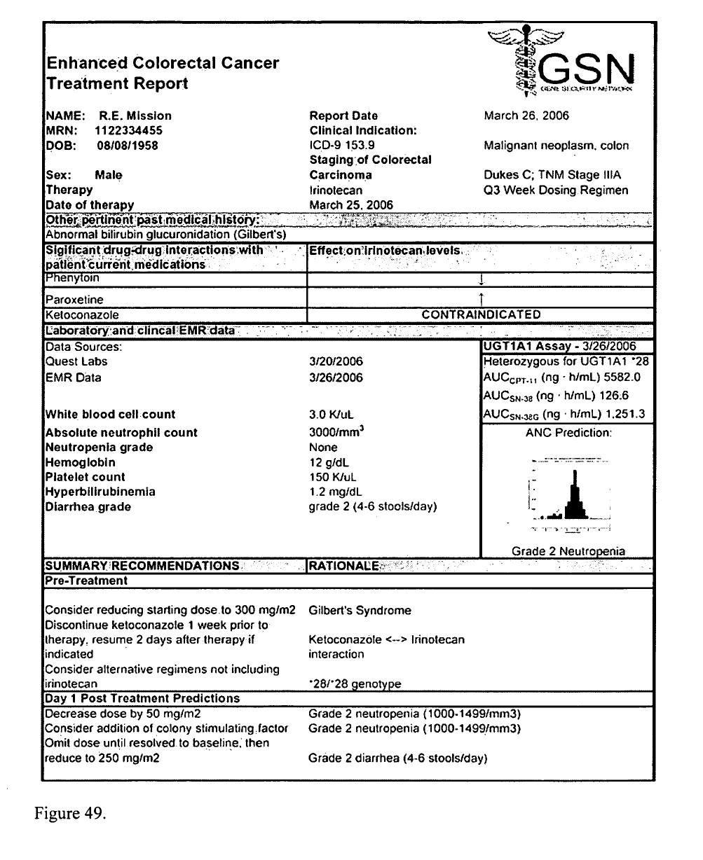

FIG. 49 is a mock-up of an enhanced report for colorectal cancer treatment with irinotecan. Prior to treatment, the report takes into account the patient's cancer stage, past medical history, current medications, and UGT1A1 genotype to recommend drug dosage. During treatment, the patient's blood counts, diarrhea grade, and irinotecan metabolite measurements (e.g. SN-38) can be monitored and used to create additional enhanced reports for treatment adjustments. Data sources and justification for recommendations are provided. Thus, the described irinotecan report will efficiently condense into an easily-readable format the information physicians need to provide the best care to their colon cancer patients and to maximize their therapeutic dose.

It should be obvious to one skilled in the art how enhanced clinical reports could be generated for individuals in other situations, and with other conditions, ailments, or diseases.

Engineering Specifications for Implementing the Ontology, Data Entry Templates and Cartridges, and Data Integration

In one embodiment of the invention, the pharmacokinetic CSO may be rendered as an XML Schema Definition document (XSD). This will contain the information necessary to generate meaningful headings in an Excel template by associating each column and each group of columns with a title element that contains a fixed XPath expression. The XPath expression will be compiled based on the selected data classes. Shown below is an XPath expressions for a column group heading (e.g. “Irinotecan: Intravenous Infusion: Recurrent Similar Events”), followed by an XPath expression for a particular column heading (e.g. “Dose Amount: mg/mˆ2”). What follows is an excerpt of some of one possible XPath document:

```
<xpathExp>
    (/DrugDosingEvent/Description/DisplayName)|(/DrugDosingEvent/
Description/DrugAdministeredToSubject)
</xpathExp>
<xpathExp>
    <appendIfNotNull>:</appendIfNotNull>/DrugDosingEvent/
Description/RouteOfAdministration>
</xpathExp>
Recurrent Similar Events
Dose Amount:
<xpathExp>../Description/DoseAmountUnits</xpathExp>
```

Implementation of the Statistical Rules for Data Validation

There are many sets of statistical rules that may be used for the purpose of data validation. In one embodiment of the invention, the statistical method DIST may be used. DIST checks the distribution of the variables only within a particular column or data class, and does not use any regression models to link variables statistically. The DIST will be used for both categorical variables and numerical variables. In both cases, variables that lie below a particular user configured probability level (e.g. 5%) will be flagged. In the case of numerical variables, a histogram will be shown, with the specified confidence bounds in blue and the outlier in red. In the case of categorical variables, a bar chart will be shown with the bar corresponding to the offending variable in red. For numerical values, the confidence bounds will be empirical bounds based on the histogram, and will not be based on fitting the data to a Gaussian distribution.

The distribution against which variables are checked will be based on the system ID that is associated with that variable, which will also be associated with a glob of XML describing that variable and stored in the database. In other words, if any changes occur in the meta-data describing a variable, a new distribution will be created for that variable. A single directory will contain a set of mat files, each of which is associated with a particular system ID. When data is submitted, if the SYSTEM ID is valid, the .mat files will be created. Otherwise, the .mat files will be loaded and augmented with the new counts. Even if the cartridge is new, data will be checked against other data in the newly submitted file. The process will be as follows:

(1) For a file submission, the matlab function Validate_Data_PharmGKB is used in which each column with a system ID will be checked for against a model (.mat file). The Interface to Validate_Data_PharmGKB is as in the following MATLAB code illustration. Code is omitted that would be obvious to one skilled in the art. For this illustration, it is assumed that the user of the template is proficient in MATLAB and Structured Query Language (SQL.)

function Validate_Data_PharmGKB(input_filename, output_filename, predict_fn, model_path, figure_output_path, fig_name, plot_flag, print_flag, remodel_flag);

% This function reads data from the input file, and a model from a .mat

% file, and determines whether the data is consistent with the prediction

% of predict_fn. If the model file does not exhist it is created. For each

% record, first check to see if it's already in the model by checking

% record_ID and value. If record is in model, remove record from model to

% validate. Once validated the record is added to the model again.

%

% inputs

% input_filename—string for text file from which input data is read. Structure of file is:

% number of rows of data

% number of columns of data

% confidence level e.g. 0.95

% IDs associated with each variables XML glob

% flag indicating num, txt, ignore (1,2,3)

% output_filename—string for text file to which output data is written; Structure of file is:

% IDs associated with each variables XML glob

% recordID for each row

% represents 1/0/-1 (yes/no/neither) for validating output

% predict_fn—string identifying the technique to be used e.g.,‘DIST’, ‘LASSO’ (only DIST supported here)

% model_path—string describing path to relevant model e.g.:

‘C:\dev\prototype\PredictionPackage\PharmGKBv1.0\Model\’

% figure_path—string describing path to where figures are plotted

% fig_name—string describing the base of the .jpg filename to which image is drawn e.g.:

‘<fig_name>_<recordID>_<systemID>.jpg’

% plot_flag—integer indicating whether to plot figure or not

% remodel_flag—flag telling program to ignore exhisting distribution and recreate from scratch

% outputs

%<file is output describing success/failure (1/0), PVALUE><graphs also output>

If the systemID is new (no mat model file exists)
the distribution will be created with all the new data
the data outside the 95% (or whatever confidence bound) will be flagged
the distribution will then be created again with all flagged data removed

If the systemID is not new, then the data for the variable will be validated against the existing distribution and added to the distribution if validated.

the user will then either change or corroborate the flagged data

individual data can be added to the distribution with a function: add_to_dist (filename_name)

The text file <file_name> records variables to be added in rows of:

record_ID1, systemID1, data1

record_ID2, systemID2, data2

If an added variable matches to a variable with a warning and the variable value is unchanged, the warning is removed and the variable is added to the distribution. If an added variable matches to a variable with a warning and the variable is changed, then the warning is removed, the variable is added to the distribution, and the whole data set corresponding to systemID is again validated.

### DEFINITIONS

GSN: Gene Security Network; the name of the company involved in the development of this invention, and the context in which this invention is being developed. The screenshots are of a particular embodiment of the invention developed specifically for Gene Security Network.
Validate: to use statistical and/or expert rules to interrogate data and uncover individual datum that are likely to be in error, flag those datum, and give a stamp of approval to the remaining data. Validation may also include steps taken by a validator to manually approve certain pieces of data.
Validator: an entity or individual who validates a given piece of information.
System ID: The System Identifier is the identifying information connected to a piece of data. It can be a synonym of a UMLS concept, a relation between two or more UMLS concepts, a concept from a CSO, a relation between two or more concepts from a CSO, or a mixture thereof.
Map: to define or discover the one-to-one correlation between a piece of information or information location in one context (for example, a database with a given format) and the corresponding piece in another context.
Cartridge: an electronic translation definition, and/or a script or program capable of implementing the defined electronic translation. The cartridge is capable of assimilating the data from one source, in one format, into the appropriate locations of a database using another format, or into newly created locations where appropriate. The cartridge may act as the root element of the CSO, and may contain one or more “column groups” and each column group must contain at least one “description field”—which provides metadata that refines the context of the column group. Each column group may also contain one or more “column field” which describes a particular column or data class that resides within the column group. The description fields for the column group provide context for the column fields that belong to that column group.
Ontology: a specification of a domain of knowledge. An ontology is a controlled vocabulary that describes concepts and the relations between them in a formal way and has a grammar for using the vocabulary terms to express something meaningful within a specified domain of interest. The ontologies created in this invention define a set of data classes which represent simple and complex concepts. Data classes can be as simple as “numeric value” for example, and as complex as whole medical procedures. Each data class can be related to another data class through a “relationship”. A pair of data classes related to each other through a relationship is called a “statement” which is itself a data class. The ontology is a complex network of these statements. The structure of one possible ontology used in this disclosure is modeled after Semantic Web specifications. See http://www.w3.org/2001/sw/
Pharmacodynamics: the body's response to a pharmaceutical agent.
DAME: data analysis and management engine
CSO: context specific ontology.
EMR: electronic medical records.
XML: extension markup language.
CUI: concept unique identifier.

What is claimed is:

1. A method for integrating genetic, phenotypic and medical data into a database according to a standardized ontology, the method consisting of:
(i) defining and creating a standardized ontology that can accommodate all of the relevant pieces of data and data fields,
(ii) generating an interface based on the standard ontology that allows an agent to describe the data fields of the input data appropriately, and then input the data,
(iii) generating a cartridge that is capable of translating the data into a format that is compliant with the standardized ontology, and
(iv) translating and loading the input data into the database.

2. A method as in claim 1, where the integrated data undergoes validation, the validation consisting of:
(i) describing a set of expectations regarding a set of input data based on statistical models and/or expert rules,
(ii) determining the likelihood of the validity of the individual pieces of input data by checking if they conform to the expectations,
(iii) flagging any pieces of data that do not conform to the expectations, and
(iv) approving any pieces of data that do conform to the expectations.

3. A method as in claim 1, where the data is subjected to a statistical analysis that allows the calculation of the likelihood of one or more phenotypic, clinical and/or medical outcomes for a particular patient given certain possible courses of treatment, and where those predictions are formulated into a report for physicians or other agents of a subject of the data.

4. A method as in claim 1, where the integrated data is computationally comparable to other related data that was collected from other sources and assimilated into the database.

5. A method as in claim 1, where the data is subjected to a statistical analysis that allows a phenotypic prediction to be made from the data.

6. A method as in claim 1, where the data is subjected to a statistical analysis that allows a clinically relevant prediction to be made from the data.

7. A method as in claim 1, where the data is used to make a prediction, and the accuracy of the prediction is quantified with a confidence estimate.

8. A method as in claim 1, where the standardized data classes are based on a set of existing standards for clinical, laboratory and genetic data.

9. A method as in claim 1, where the data is generated in the context of a clinical trial.

10. A method as in claim 1, where the data is generated in the context of diagnostic screening.

11. A method as in claim 2, where the validation includes a step that allows a user to act upon the status of a piece of flagged data, the actions taken from a list comprising: to override the flagging and approve the datum, to correct the datum, to remove the datum from the dataset, to resubmit the datum for validation, and combinations thereof.

12. A method as in claim 2, where the statistical model that shows the highest accuracy during a training of the model with a second set of data is selected from a plurality of statistical models in order to make the most accurate prediction.

13. A method as in claim 2, where the statistical model is trained on sparse data using one or more shrinkage functions.

14. A method as in claim 2, where an association is maintained between certain pieces of validated data and the validator of that piece of data, and where a record indicating the reliability of the validator is made available to entities who are in a position to make clinical or market decisions based on the validated data.

15. A method as in claim 2, wherein the data validation is re-examined using the latest available computer-executable rules and data, and where data managers are notified whenever the status of validation pertaining to a given datum change.

16. A method as in claim 3, where the data analyses are frequently re-examined, and where a new report is generated when one or more predictions in the report change significantly due to pertinent new information and/or data becoming available.

17. A method as in claim 3, where the report is generated automatically at periodic time intervals.

18. A computer implemented method configured to perform the method described in claim 1.
